(19) United States
(12) Patent Application Publication
(10) Pub. No.: US 2025/0154215 A1
(43) Pub. Date: May 15, 2025

(54) PHARMACEUTICAL COMPOSITION FOR TREATING COLITIS OR COLORECTAL CANCER COMPRISING COLON-TARGETED S100A8/A9-DERIVED SPECIFIC PEPTIDE

(71) Applicant: Industry-University Cooperation Foundation Hanyang University ERICA Campus, Ansan-si (KR)

(72) Inventors: Chul-Su YANG, Ansan-si (KR); Euni CHO, Ansan-si (KR); Seok-Jun MUN, Ansan-si (KR)

(73) Assignee: Industry-University Cooperation Foundation Hanyang University ERICA Campus, Ansan-si (KR)

(21) Appl. No.: 18/883,449

(22) Filed: Sep. 12, 2024

(30) Foreign Application Priority Data
Nov. 9, 2023 (KR) 10-2023-0154846
Dec. 20, 2023 (KR) 10-2023-0186669

(51) Int. Cl. C07K 14/52; A61K 38/00; A61P 1/00; A61P 35/00

(52) U.S. Cl. CPC C07K 14/52; A61P 1/00; A61P 35/00; A61K 38/00; C07K 2319/00

(57) ABSTRACT

The present invention relates to a dual PRR-inhibiting peptide system consisting of TLR4- and RAGE-inhibiting motifs derived from S100A8 and S100A9 and conjugated with a CT peptide for colon-specific delivery. In human-derived monocyte THP-1 and mouse bone marrow-derived macrophages (BMDMs), S100A8/A9-derived peptides including TLR4 and RAGE-interacting motifs inhibit the binding of S100 to TLR4 or RAGE and the activation of the NLRP3 inflammasome in a dose-dependent manner. When the peptide according to the present invention is injected into mouse models of acute and chronic colitis, survival is significantly increased, and pathological damage to the colon is alleviated. In a mouse model of colitis-related colorectal cancer, when the peptide according to the present invention is injected, body weight is increased, tumor burden in the distal colon is decreased, and histological colon damage is significantly alleviated. When the peptide according to the present invention is injected into an oxaliplatin-resistant CRC xenograft mouse model, EMT-associated markers are reduced in tumor tissues and tumor growth is significantly inhibited.

### CROSS-REFERENCE TO RELATED APPLICATION

This application claims priority to Korean Patent Application No. 10-2023-0154846, filed on Nov. 9, 2023, and Korean Patent Application No. 10-2023-0186669, filed on Dec. 20, 2023, the disclosures of which are incorporated herein by reference in their entireties.

### INCORPORATION BY REFERENCE OF SEQUENCE LISTING

The content of the electronically submitted sequence listing, file name: Q302066_SEQ_LIST_AS_FILED.xml; size: 13.2 KB; and date of creation: Sep. 11, 2024, filed herewith, is incorporated herein by reference in its entirety.

### BACKGROUND

### 1. Field of the Invention

The present invention relates to a pharmaceutical composition for treating colitis or colorectal cancer, including a colon-targeted S100A8/A9-derived specific peptide.

### 2. Discussion of Related Art

Inflammatory bowel disease (IBD), such as ulcerative colitis and Crohn's disease, is a chronic immune-mediated inflammatory disease of the intestinal tract. IBD is characterized by long-term intestinal inflammation, which leads to the formation of intractable ulcers and severe destruction of the intestinal mucosa. Although the underlying pathophysiology of IBD is not fully understood, it was found that dysregulated cytokines and immune cells and intestinal microbiota imbalances are associated with persistent inflammation. Patients with IBD are at higher risk of developing colitis-related colorectal cancer (CRC), with an incidence rate of 15% to 20%. This indicates that there is association between chronic inflammation and cancer development.

To date, the most successful strategy for treating IBD has been the use of biologics that target the excessive activity of the adaptive immune system. A representative drug of this type is infliximab, which is a monoclonal antibody designed to inhibit tumor necrosis factor α (TNFα). Nonetheless, studies have shown that anti-TNFα treatment is ineffective in more than one-third of patients with IBD. Therefore, there is an urgent need to develop new therapeutic approaches for IBD.

Meanwhile, one of the current therapeutic strategies used to treat CRC includes the administration of chemotherapy agents (e.g., 5-fluorouracil, irinotecan, and oxaliplatin) and/or targeted therapies (e.g., cetuximab and bevacizumab). For terminal and progressive metastatic diseases, radiotherapy may be optionally included. However, long-term use of this treatment strategy eventually leads to the development of resistance in all CRC cells. As the incidence of IBD and CRC continues to increase worldwide, there is an urgent need for more effective treatment approaches that can effectively address resistance and immunogenicity issues.

In addition to acting as alarmins, S100A8 and S100A9 act as damage associated molecular patterns (DAMPs), which activate the innate immune system through binding to pattern recognition receptors (PRRs) such as the receptor for advanced glycation end products (RAGE) and toll-like receptor 4 (TLR4). This results in the activation of downstream transcription factors such as NF-κB and the production of pro-inflammatory cytokines. Many studies have shown that serum levels of S100A8/A9 are elevated in numerous inflammatory diseases, including IBD, rheumatoid arthritis, psoriasis, and vasculitis. S100A8 and S100A9 are also known to accelerate the progression of colitis by disrupting intestinal mucosal homeostasis and triggering pro-inflammatory signaling cascades. DAMPs also form a microenvironment that promotes the development of CRC, possibly by contributing to uncontrolled inflammatory responses. In other words, it was found that chronic inflammation that is initiated, sustained, and amplified by the S100A8/A9-PRR axis is involved in the association between inflammation and cancer (CRC). Therefore, to treat IBD and CRC, it may be therapeutically useful to treat IBD and CRC with therapeutics having targeting potential.

Colon-specific drug delivery, which enables targeted drug delivery to the colon, holds considerable promise for local treatment of a variety of intestinal diseases, including ulcerative colitis, Crohn's disease, and colorectal cancer. Furthermore, in the context of IBD, implementing local blockade specifically on the intestinal mucosa may be a promising therapeutic strategy to enhance drug efficacy and reduce drug toxicity. One study showed that the inflamed colon may be targeted by a 12-residue peptide (TWYKIAFQRNRK)(SEQ ID NO: 1). Motivated by this, the present inventors previously developed a colon-specific delivery system with the ability to modulate the NOD-like receptor protein 3 (NLRP3) inflammasome, which successfully targeted the colon.

### NON-PATENT DOCUMENT

Kim, J.-S. et al., Colon-Targeted eNAMPT-Specific Peptide Systems for Treatment of DSS-Induced Acute and Chronic Colitis in Mouse. Antioxidants, 2022, 11, 2376.

### SUMMARY OF THE INVENTION

The present invention is directed to providing a pharmaceutical composition for treating colorectal cancer or colitis.

The present invention relates to a polypeptide in which four peptides consisting of amino acid sequences represented by SEQ ID NO: 2 to SEQ ID NO: 5 are linked through peptide bonds. In one embodiment of the present invention, the four peptides are linked with a linker, in another embodiment of the present invention, the linker is (G)ₙ, (A)ₙ, or (G₄S)ₘ, where m is an integer from 1 to 3, and n is an integer from 2 to 8, in still another embodiment of the present invention, the amino acid sequence is (SEQ ID NO: 2)-(linker)-(SEQ ID NO: 3)-(linker)-(SEQ ID NO: 4)-(linker)-(SEQ ID NO: 5), in yet another embodiment of the present invention, SEQ ID NO: 1 is further linked to an N-terminus or C-terminus by a peptide bond, and in yet another embodiment of the present invention, disclosed is a polypeptide represented by (SEQ ID NO: 1)ₐ-(linker)-{(SEQ ID NO: 2)-(linker)-(SEQ ID NO: 3)-(linker)-(SEQ ID NO: 4)-(linker)-(SEQ ID NO: 5)}_{b}, wherein a is an integer from 1 to 7, and b is an integer from 1 to 5.

One embodiment of the present invention discloses a polypeptide including SEQ ID NO: 6. In one embodiment of the present invention, SEQ ID NO: 1 is further linked to an N-terminus or C-terminus by a peptide bond, in another embodiment of the present invention, the polypeptide is represented by (SEQ ID NO: 1)ₐ-(linker)-(SEQ ID NO: 6)_{b}, where a is an integer from 1 to 7, and b is an integer from 1 to 5, in still another embodiment of the present invention, the linker is (G)ₙ, (A)ₙ, or (G₄S)ₘ, where m is an integer from 1 to 3, and n is an integer from 2 to 8, and in yet another embodiment of the present invention, the polypeptide consists of an amino acid sequence represented by SEQ ID NO: 7.

The present invention discloses a pharmaceutical composition for treating colitis or colorectal cancer, including the above polypeptide.

One embodiment of the present invention provides a nucleic acid molecule encoding a polypeptide, a recombinant vector including the nucleic acid molecule, and a recombinant strain including the nucleic acid molecule or recombinant vector.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1A-1D show that the interaction between S100A8 or A9 and toll-like receptor 4 (TLR4) or receptor for advanced glycation end products (RAGE) is increased in inflammation-related colon disease in mice. FIGS. 1A and 1B show that 293T cells were transfected with Myc-TLR4 (FIG. 1A, left) or Myc-RAGE (FIG. 1B, left) and HA-S100A8 or His-S100A9. The 293T cells were used for immunoprecipitation (IP) including αMyc, and then for immunoblotting (IB) including αHA or αHis. FIG. 1A shows that whole cell lysates (WCLs) were used for IB including αHA, αHis, αMyc, or αActin. Bone marrow-derived macrophages (BMDMs) were incubated with rHA-S100A8 or rHis-S100A9 (5 μg/ml) for 10 minutes. The BMDMs were subjected to IP using αTLR4 (FIG. 1A, right) or αRAGE (FIG. 1B, right), and then subjected to IB using αHA, αHis, αTLR4, or αRAGE. The WCLs were used for IB including αHA, αHis, αTLR4, αRAGE, or αActin. In FIG. 1C, on the left, relative expression of S100A8 or S100A9 in colons of normal, acute colitis, chronic colitis, and colorectal cancer mice was determined, and biological replicates (n=10) were used for each condition. On the right, IB was performed on sera from normal, acute colitis, chronic colitis, and colorectal cancer mice using αS100A8 or αS100A9. Coomassie blue (CB)-stained gels show consistent loading of serum proteins. Data from 3 out of 10 samples is shown. FIG. 1D shows that colon lysates from normal, acute colitis, chronic colitis, and colorectal cancer mice were subjected to IP using αTLR4 or αRAGE, and then subjected to IB using αS100A8 or αS100A9. The WCLs were used for IB including αS100A8, αS100A9, αTLR4, αRAGE, or αActin. Data is representative of seven independent experiments with similar results (FIGS. 1A-1D);

FIGS. 2A-2D show the discovery and design of S100A8/A9 peptides that inhibit essential domain binding to TLR4 and RAGE. FIGS. 2A and 2B show schematic diagrams of the structures of S100A8 (FIG. 2A) and S100A9 (FIG. 2B) (top). 293T cells were transfected with GST-S100A8 (left), S100A9 (right) or their mutants and Myc-TLR4 or Myc-RAGE. The 293T cells were subjected to glutathione S-transferase (GST) pulldown, and then subjected to IB using αMyc or αGST. WCLs were used for IB including αMyc, αGST, or αActin. Bottom, peptide design of S100A8 and S100A9 including TLR4 and the RAGE-interacting motif of S100A8 and S100A9 sequences (here, the aa sequence of the motif is selected by the shared aa between the human and mouse). FIG. 2C shows that BMDMs were incubated with rHA-S100A8 or rHis-S100A9 (5 μg/ml) for 10 minutes in the presence of S100A8 or S100A9 peptides for one hour. The BMDMs were subjected to IP using αHA or αHis, and then subjected to IB using αTLR4 or αRAGE. The WCLs were used for IB including αHA, αHis, αTLR4, αRAGE, or αActin. FIG. 2D shows that THP-1 or BMDMs were stimulated with lipopolysaccharide (LPS) (100 ng/ml) for one hour in the presence of S100A8 or S100A9 peptides for 18 hours. The BMDMs were subjected to IP using αTLR4 or αRAGE, and then subjected to IB using αS100A8 or αS100A9. The WCLs were used for IB including αTLR4, αRAGE, αS100A8, αS100A9, or αActin. Data is representative of seven independent experiments with similar results (FIGS. 2A-2D);

FIG. 3 shows that S100A8/A9 peptides effectively block intracellular uptake in macrophages. BMDMs were incubated with Texas Red-conjugated rHA-S100A8 or rHis-S100A9 (5 μg/ml) for 10 minutes in the presence of S100A8 or S100A9 peptides for one hour. The BMDMs were analyzed for Texas Red+ cell numbers through fluorescence-activated cell sorting (FACS). Data is representative of three independent experiments with similar results. Data is shown as the mean±standard deviation (SD) of three experiments. MFI, mean fluorescence intensity;

FIGS. 4A-4E show that S100A8/A9 peptide inhibits the activation of the nucleotide-binding oligomerization domain (NOD)-like receptor protein 3 (NLRP3) inflammasome. FIG. 4A shows that LPS-primed THP-1 or BMDMs were treated with S100A8 or S100A9 peptides for one hour and then activated with ATP (1 mM) for 30 minutes, and the subjected to IP using αTLR4 or αRAGE and IB using αS100A8 or αS100A9. WCLs were used for IB including αTLR4, αRAGE, αS100A8, αS100A9, or αActin. FIG. 4B shows schematic illustration of S100A8/9 peptide design (top). LPS-primed THP-1 or BMDMs were treated with S100A8/9 peptide (0.01, 0.1, 1 μM) for one hour and then activated with ATP for 30 minutes. The experimental conditions follow the same pattern as described in FIG. 4A. FIG. 4C shows that BMDMs were subjected to IP using αNLRP3 or αASC and then to IB using αASC or αNLRP3. WCLs were used for IB including αNLRP3, αASC, or αActin. FIGS. 4D and 4E show that LPS-primed BMDMs were treated with 9R-S100A8/9 peptide (0.1 μM) for one hour and stimulated with ATP, nigericin, monosodium urate (MSU), alum, poly(dA:dT), flagellin or liver transplantation (LTx) for one hour or with dextran sodium sulfate (DSS) for 18 hours. FIG. 4D shows ELISA for IL-1β and IL-18. FIG. 4E shows IB of supernatant (SN) including αIL-1β p17, αIL-18 p18, or αCasp1 p10, and WCLs including αPro-IL-1β, αPro-IL-18, αPro-Casp1, or αActin. Data is shown as the mean±SD of four experiments (FIG. 4D). Statistical significance was determined by Student's t-test with Bonferroni correction (***p<0.001) compared to untreated peptide (FIG. 4D). Data is representative of seven independent experiments with similar results (FIGS. 4A-4C and FIG. 4E);

FIGS. 5A and 5B show the drug-like properties of rCT-S100A8/A9. FIG. 5A shows the design of rCT-S100A8/9 (top). Bacteria-purified 6×His-rCT-S100A8/9, rVehicle or rCT was analyzed by Coomassie staining or IB using αHis (bottom, left). BMDMs were incubated with rVehicle, rCT, or rCT-S100A8/9 for the indicated times and at the indicated concentrations, and then cell viability was measured by MTT assay (bottom, right). FIG. 5B shows the results of in vitro plasma stability, microsomal stability, cytochrome (CYP) inhibition, and pharmacokinetic (PK) profiles of S100A8/A9 peptide and rCT-S100A8/A9 protein. HLM, human liver microsomes; CYP, cytochrome P450. aTmax, arrival time; AUClast, total area under the plasma concentration-time curve from time 0 to the time of the last measurement; Cmax, maximum plasma concentration;

FIGS. 6A-6D show the effect of rCT-S100A8/A9 peptide on colon targeting. FIG. 6A shows DSS-induced model for colitis. Mice were exposed to 3% DSS for six days and evaluated on day 6 (left). For IB with αITGA6, αITGB1, or αActin, multiple organ harvests were used and ITGA6+ or ITGB1+ cell numbers were analyzed through FACS. FIG. 6B shows scheme of acute colitis model transduced with Lenti-shNS or Lenti-shITG virus and treated with 3% DSS. Intraperitoneal administration of rCT-S100A8/9 conjugated with Cy5.5 for one hour and colon harvest. Colon harvests were used for IB including αITGA6, αITGB1, or αActin and ITGA6+ or ITGB1+ cell numbers were analyzed through FACS. FIG. 6C shows that multiple organ harvests were used and rCT-S100A8/9-Cy5.5+ cell numbers were analyzed through FACS. FIG. 6D shows pharmacodynamics of rCT-S100A8/9 in DSS-induced colitis model. Mice were treated with 3% DSS for six days and administered 1, 2, or 3 doses of Texas Red conjugated with rCT-S100A8/9. Colons were harvested in a time-dependent manner and analyzed for Texas Red+ cell numbers through FACS. Data is representative of three independent experiments with similar results. Data is shown as the mean±SD of three experiments;

FIGS. 7A-7H show that rCT-S100A8/A9 exhibited a therapeutic effect against DSS-induced acute and chronic colitis in mice. FIG. 7A shows schematic illustration of a 3% DSS-treated acute colitis model treated with rCT-S100A8/9 (50 μg/kg) (top). The survival of mice was monitored for 21 days. Mortality was measured for n=15 mice per group (bottom). FIG. 7B shows that weight loss and colitis scores were obtained from clinical variables (weight loss, stool consistency, bleeding) (n=10). FIG. 7C shows that the survival of S100A8−/− and S100A8−/− mice compared to wild-type mice was monitored for 21 days (n=15 mice per group). FIG. 7D shows FACS analysis for superoxide (top) and mitochondrial reactive oxygen species (mitoROS) (bottom). Quantitative analysis of the mean fluorescence intensity of ROS (box). FIG. 7E shows schematic illustration of chronic colitis model treated with 2.5% DSS with rCT-S100A8/9 (50 μg/kg) (top). Middle, the survival of mice was monitored for nine weeks. Mortality was determined for n=15 mice per group. Bottom, weight loss of rVehicle, rCT or rCT-S100A8/9 in mice (n=15). FIG. 7F shows a representative image of H&E staining of the colon (left) (n=10). Histopathology scores were obtained from hematoxylin and eosin (H&E) staining as described in Methods (Materials and Methods) determined in 2.5% DSS-treated mice treated with rVehicle, rCT, or rCT-S100A8/9 (right). FIG. 7G shows the survival of S100A8−/− and S100A8−/− mice compared to wild-type mice was monitored for nine weeks (n=15 mice per group). FIG. 7H shows FACS analysis for mitoROS. Quantitative analysis of the mean fluorescence intensity of ROS (box). Statistical differences compared to rVehicle-treated mice (FIGS. 7A and 7E) or rCT-treated mice (FIGS. 7C and 7G) are shown (log-rank test). Data is representative of two independent experiments with similar results (FIGS. 7A, 7C, 7E, and 7G). Statistical significance was determined by Student's t-test with Bonferroni correction (***P<0.001) compared to rVehicle (FIG. 7F);

FIGS. 8A-8G show the effect of rCT-S100A8/A9 against DSS-induced acute and chronic colitis in mice. The experimental conditions followed the protocol described in FIG. 5A. FIG. 8A shows images (top) and the length (bottom) of the colon of 3% DSS-induced acute colitis mice treated with rVehicle, rCT, or rCT-S100A8/9 (n=10). FIG. 8B shows representative imaging of H&E staining of the colon (left) (n=10). Histopathology scores were obtained through H&E staining as described in Methods (Materials and Methods) determined in 3% DSS-treated mice treated with rVehicle, rCT, or rCT-S100A8/9 (right). FIG. 8C shows that colons were used for IP with αTLR4, αRAGE, αNLRP3, or αASC, and then for IB with αS100A8, αS100A9, αNLRP3, or αASC. WCLs were used for IB including αTLR4, αRAGE, αS100A8, αS100A9, αNLRP3 or αASC, αHis or αActin. FIG. 8D shows cytokine and myeloperoxidase (MPO) activity levels in colon homogenates (n=10). FIG. 8E shows images (top) and the length (bottom) of the colon of 2.5% DSS-induced chronic colitis mice treated with rVehicle, rCT, or rCT-S100A8/9. (f and g) Macrophage polarization markers using M1 (CD86, iNOS, or CD80) or M2 (CD163, Arg1, or CD206) were measured by quantitative real-time PCR in BMDMs (FIG. 8F) and colon lysates (FIG. 8G). Statistical significance was determined by Student's t-test with Bonferroni correction (*P<0.05; **P<0.01; ***P<0.001) compared to rVehicle;

FIGS. 9A-9D show that rCT-S100A8/A9 has a therapeutic effect against azoxymethane (AOM)/DSS-induced colitis-associated colorectal cancer in mice. FIG. 9A shows schematic illustration of AOM (10 mg/kg)/DSS (2.5%)-induced colitis-associated colorectal cancer model treated with rCT-S100A8/9 (50 μg/kg) (top). Weight of mice compared to baseline during AOM/DSS administration. In this experiment, weight loss is a surrogate indicator of colitis severity. Data is representative of two independent experiments with similar results. FIG. 9B shows macroscopic findings of the distal colon. Note the higher tumor burden in the distal colon/rectum and the characteristic wrinkled texture of the proximal colon with little tumor growth. FIG. 9C shows representative distribution of the average number of tumors per mouse treated with AOM/DSS. Most tumors are located in the distal colon, and their sizes are less than 2 mm. Data is shown as the mean±SD of six experiments. FIG. 9D shows representative histology of tumors resulting from AOM/DSS administration in the distal colon (n=10). H&E and β-catenin stained slides show dysplastic changes similar to human colonic adenocarcinoma;

FIGS. 10A-10B show that differentially expressed genes in colonic lysates are selected through an RT² profiler PCR array test. To identify the most significant differentially expressed genes (DEGs), RT-qPCR was performed on more mouse samples. Horizontal coordinates represent fold differences, and the threshold was set to |log 2 fold change (FC)|>2. Heatmaps visualize the fold changes in expression between selected groups for all genes in an array in the context of the array layout;

FIGS. 11A-11F show rCT-S100A8/A9 ameliorated oxaliplatin-resistant HCT116 xenograft tumor growth. FIG. 11A shows that parental and oxaliplatin-resistant HT29, HCT116, and SW480 colorectal cancer cell lines were treated with various concentrations of oxaliplatin or rCT-S100A8/9 for 24 hours, and their cell viability was analyzed. Each vertical bar represents the mean±SD of cell viability at each dose. Data is shown as the mean±SD of six experiments. FIGS. 11B and 11C show that parental cells or HCT116 of OxaR were stimulated with rS100A8/9 (10 μg/ml) for one hour in the presence of rCT-S100A8/9 (10, 20, and 50 μg/ml) for 30 minutes. FIG. 11B shows that HCT116 was subjected to IP using αTLR4 or αRAGE and then to IB using αS100A8 or αS100A9. WCLs were used for IB including αTLR4, αRAGE, αS100A8, αS100A9, or αActin. FIG. 11C shows IB including αE-cadherin, αN-cadherin, αVimentin, αP-EGFR, αP-Akt, αP-NF-kB p65, or αActin. FIG. 11D shows schematic illustration of xenograft model treated with rCT-S100A8/9 (top). HCT116 cells were injected subcutaneously into the flanks of nude mice. Tumor length and width were measured using calipers, and tumor volume was calculated every 3 days for 30 days. Representative images of tumors from mice treated with rCT-S100A8/9 on day 15 (middle). Statistical significance was determined by two-way analysis of variance (ANOVA) with Tukey's post hoc test. *P<0.05, ***P<0.001 compared to rVehicle. FIG. 11E shows that on day 21 after administration, mice were euthanized, and tumor lysates were used for IP including αTLR4 or αRAGE and then used for IB including αS100A8 or αS100A9. WCLs were used for IB including αTLR4, αRAGE, αS100A8, αS100A9, or αActin. FIG. 11F shows representative histology of tumors (n=10). Tumors were stained with H&E. Data is representative of seven independent experiments with similar results (FIGS. 11B, 11C, and 11E);

FIGS. 12A-12E show biological effects of rCT-S100A8/9 in tumor-bearing mice. FIG. 12A shows that both parental cells and oxaliplatin/doxorubicin or oxaliplatin/5-Fu resistant HT29 and HCT116 colorectal cancer cell lines were treated with various concentrations of rCT-S100A8/9 for 24 h, and their cell viability was analyzed. Each vertical bar represents the mean±SD of cell viability at each dose. Data is shown as the mean±SD of six experiments. FIG. 12B shows pharmacokinetics and drug distribution of rCT-S100A8/9 in tumor-bearing mice. HCT116 cells were injected subcutaneously into the flanks of BALB/c mice as described in the Methods section. Mice were administered one or two intraperitoneal doses and sacrificed on day 5. Their expression was assessed by IB in various organs of mice. WCLs were used for IB including αActin. Data is representative of four independent experiments with similar results. FIG. 12C shows representative histology of tumors (n=10). E-cadherin, N-cadherin, and β-catenin staining of the tumors. FIG. 12D shows humoral immune response in BALB/C mice. Mice were immunized by each route (i.v. or i.p.) every 3 days, and blood was collected on day 40. IgG levels specific for rCT-S100A8/9 or ovalbumin (OVA) were measured using ELISA. Values represent the mean optical density (OD) and standard deviation at 450 nm. Each group included five mice. FIG. 12E shows cytokine response to rCT-S100A8/9 in tumor-bearing mice. HCT116 cells were injected subcutaneously into the flanks of BALB/c mice as described in the Methods section. Serum cytokine levels were measured 30 days after administration. Each group included 10 mice; and

FIG. 13 shows that DEGs in tumor lysates are selected through an RT² profiler PCR array test. To identify the most important DEGs, RT-qPCR was performed on more mouse samples. Horizontal coordinates represent fold differences, and the threshold was set to |log 2 fold change (FC)|>2. Heatmaps visualize the fold changes in expression between selected groups for all genes in an array in the context of the array layout.

### DETAILED DESCRIPTION OF EXEMPLARY EMBODIMENTS

Hereinafter, the present invention will be described in detail through embodiments of the present invention with reference to the attached drawings. However, the following embodiments are presented as examples of the present invention, and when it is judged that a detailed description of a technology or configuration well known to those skilled in the art may unnecessarily obscure the gist of the present invention, the detailed description may be omitted, and the present invention is not limited thereby. Various modifications and applications of the present invention are possible within the scope of the claims described below and the equivalents interpreted therefrom.

In addition, the terms used herein are terms used to appropriately express preferred embodiments of the present invention, and may vary depending on the intention of the user or operator or the practices of the field to which the present invention pertains. Therefore, definitions of these terms should be made based on the content throughout the present invention. Throughout the present invention, when a part is said to “include” a certain component, unless specifically stated to the contrary, this means that it may further include other components rather than excluding other components.

All technical terms used in the present invention, unless otherwise defined, are used with the same meaning as commonly understood by one of ordinary skill in the art in the field related to the present invention. In addition, although preferred methods and samples are described in the present invention, similar or equivalent methods and samples are also included in the scope of the present invention. The contents of all publications incorporated herein by reference are incorporated in the present invention.

The present invention provides a fusion protein in which fragments derived from S100A8 and S100A9 are fused. More specifically, the present invention provides a fusion protein in which a TLR4-inhibiting motif (SEQ ID NO: 2 and SEQ ID NO: 4) and a RAGE-inhibiting motif (SEQ ID NO: 3 and SEQ ID NO: 5) respectively present in S100A8 and S100A9 are fused. One embodiment of the present invention provides a fusion protein including an amino acid represented by (SEQ ID NO: 2)-(linker)-(SEQ ID NO: 3) in which a TLR4-inhibiting motif and a RAGE-inhibiting motif derived from S100A8 are fused. Another embodiment of the present invention provides a fusion protein including an amino acid represented by (SEQ ID NO: 4)-(linker)-(SEQ ID NO: 5) in which a TLR4-inhibiting motif and a RAGE-inhibiting motif derived from S100A9 are fused. Still another embodiment of the present invention provides a fusion protein including an amino acid represented by (SEQ ID NO: 2)-(linker)-(SEQ ID NO: 3)-(linker)-(SEQ ID NO: 4)-(linker)-(SEQ ID NO: 5). Yet another embodiment of the present invention provides a fusion protein including an amino acid represented by (SEQ ID NO: 1)ₐ-(linker)-{(SEQ ID NO: 2)-(linker)-(SEQ ID NO: 3)-(linker)-(SEQ ID NO: 4)-(linker)-(SEQ ID NO: 5)}_{b}, wherein a is an integer from 1 to 7 and b is an integer from 1 to 5. One embodiment of the present invention provides a fusion protein including SEQ ID NO: 6. One embodiment of the present invention provides a fusion protein in which SEQ ID NO: 1 is further linked to an N-terminus or C-terminus by a peptide bond, and another embodiment of the present invention provides a fusion protein including an amino acid sequence represented by (SEQ ID NO: 1)ₐ-(linker)-(SEQ ID NO: 6)_{b}, wherein a is an integer from 1 to 7 and b is an integer from 1 to 5. Still another embodiment of the present invention provides a fusion protein consisting of an amino acid represented by SEQ ID NO: 7. Sequences of the peptides used in the present invention are shown in Table 1 below.

TABLE 1

| SEQ ID | | |
|---|---|---|
| NO: | Sequence | Description |
| 1 | TWYKIAFQRNRK | Colon targeting |
| | | sequence |
| 2 | FLVIK | S100A8_TLR4- |
| | | inhibiting sequence |
| 3 | AHKSHK | S100A8_RAGE- |
| | | inhibiting sequence |
| 4 | ITIITF | S100A9_TLR4- |
| | | inhibiting sequence |
| 5 | GHHGG | S100A9_RAGE- |
| | | inhibiting sequence |
| 6 | FLVIKGGAHKSHKGGITIITFGGGHHGG | S100A8/A9 |
| 7 | TWYKIAFQRNRKTWYKIAFQRNRKTWYKIAFQR | rCT-S100A8/A9 |
| | NRKTWYKIAFQRNRKTWYKIAFQRNRKGGFLVI | |
| | KGGAHKSHKGGITIITFGGGHHGGFLVIKGGAHK | |
| | SHKGGITIITFGGGHHGGFLVIKGGAHKSHKGGITI | |
| | ITFGGGHHGG | |
| 8 | MPSELEKALSNLIDVYHNYSNIQGNHHALYKNDF | S100A8 |
| | KKMVTTECPQFVQNINIENLFRELDINSDNAINFE | |
| | EFLAMVIKVGVASHKDSHKE | |
| 9 | MANKAPSQMERSITTIIDTFHQYSRKEGHPDTLSK | S100A9 |
| | KEFRQMVEAQLATFMKKEKRNEALINDIMEDLD | |
| | TNQDNQLSFEECMMLMAKLIFACHEKLHENNPR | |
| | GHGHSHGKGCGK | |

The linker may increase the flexibility of the fusion protein without interfering with the structure of each component within the fusion protein. In some embodiments, the linker moiety is a peptide linker having a length of 2 to 100 amino acids. Exemplary linkers include linear peptides with at least two amino acid residues, such as Gly-Gly, Gly-Ala-Gly, Gly-Pro-Ala, Gly (G)ₙ, and Gly-Ser (GS) linkers. GS linkers described herein include, but are not limited to, (GS)ₙ, (GSGSG)ₙ(SEQ ID NO: 10), (G₂S)ₙ, G₂S₂G, (G₂SG)ₙ, (G₃S)ₙ, (G₄S)ₙ, (GGSGG₎Gₙ (SEQ ID NO: 11), GSG₄SG₄SG (SEQ ID NO: 12), and (GGGGS)ₙ (SEQ ID NO: 13), where n is 1 or more. One example of a (G)ₙ linker includes a G₉ linker, and examples of a (GGGGS)ₙ (SEQ ID NO: 13) linker include a GGGGS (SEQ ID NO: 13) or (GGGGS)₃ (SEQ ID NO: 14) linker. Suitable linear peptides include polyglycine, polyserine, polyproline, polyalanine, and oligopeptides consisting of alanyl and/or seryl and/or prolyl and/or glycyl amino acid residues. Linker moieties may be used to link components of the fusion proteins disclosed herein. The present invention discloses a pharmaceutical composition for treating colitis or colorectal cancer, including the above fusion protein.

One embodiment of the present invention provides a nucleic acid molecule encoding a fusion protein or a recombinant vector including the nucleic acid molecule, and a recombinant strain including the nucleic acid molecule or the recombinant vector.

The present inventors found that serum levels of S100A8 and S100A9 were elevated and their binding to pattern recognition receptors (PRRs) such as toll-like receptor 4 (TLR4) and the receptor for advanced glycation end products (RAGE) was significantly increased in both colitis and colitis-associated colorectal cancer mouse models. The present invention provides a fusion peptide (rCT-S100A8/A9) with dual PRR-inhibiting ability consisting of TLR4- and RAGE-inhibiting motifs derived from S100A8 and S100A9 and conjugated with a CT peptide (TWYKIAFQRNRK) (SEQ ID NO: 1) for colon-specific delivery.

In human-derived monocyte THP-1 and mouse bone marrow-derived macrophages (BMDMs), S100A8/A9-derived peptides including TLR4 and RAGE-interacting motifs dose-dependently inhibited the binding of S100 to TLR4 or RAGE and the activation of the NOD-like receptor protein 3 (NLRP3) inflammasome. The present inventors confirmed that rCT-S100A8/A9 has appropriate drug-like properties including in vitro stability, pharmacokinetic (PK) properties, and pharmacological activity. In mouse models of dextran sodium sulfate (DSS)-induced acute and chronic colitis, when rCT-S100A8/A9 was injected, survival rates were significantly increased, and pathological damage to the colon was alleviated. In an azoxymethane (AOM)/DSS-induced colitis-associated colorectal cancer (CAC) mouse model, when rCT-S100A8/A9 was injected, body weight was increased, tumor burden in the distal colon was decreased, and histological colonic damage was significantly alleviated. In oxaliplatin-resistant CRC xenograft mouse models, when rCT-S100A8/A9 was injected, epithelial-mesenchymal-transition (EMT)-associated markers in tumor tissue were reduced and tumor growth was significantly inhibited.

The marked upregulation of S100A8 and S100A9 may be a cause of the abnormal activation of PRR signaling, which induces a vicious cycle of inflammatory responses and worsens colon-related diseases. The present inventors discovered that serum S100A8 and S100A9 were increased in DSS-induced acute and chronic colitis and even in an AOM/DSS-induced CRC model of mice in which there were significantly increased interactions with PRRs (TLR4 and RAGE). Here, the present inventors prepared an rCT-S100A8/A9 fusion peptide, which is capable of targeting the colon and blocking S100 (A8/A9)-PRR (TLR4/RAGE) interactions. rCT-S100A8/A9 in which a colon-targeting peptide was conjugated with TLR4/RAGE-inhibiting motifs derived from S100A8 and S100A9 successfully inhibited extracellular S100A8/A9-TLR4/RAGE interactions in mice, thereby reducing colonic inflammation and disease severity in IBD-related mouse models including DSS-induced acute/chronic colitis mice and AOM/DSS-induced CRC mice. In addition, the therapeutic potential of targeting S100-PRR interactions using rCT-S100A8/A9 was demonstrated in the context of OxaR xenografts. This approach has shown promise in reducing EMT-related markers and tumor volume. In summary, the present invention establishes proof of concept for targeting S100-PRR interactions as a strategy to prevent chronic intestinal inflammation with potential relevance to both IBD and CRC.

TLR4 and RAGE, which are important innate immune sensors, are actually expressed in a variety of cells, including immune cells and cancer cells. These PRRs are already associated with sterile inflammation in the absence of microbial ligands. Dysregulation of these PRRs is associated with poor prognosis in a variety of diseases, including infectious diseases, chronic inflammatory diseases, and certain types of cancer. In inflammatory conditions, expression and activation by damage-associated molecular patterns (DAMPs) are often upregulated, enhancing immune cell activation and pro-inflammatory cytokine production. In the tumor microenvironment, S100A8 and S100A9 may stimulate TLR4/RAGE signaling in cancer to promote tumor cell proliferation, survival, invasion, angiogenesis, and immune evasion. In particular, the expression levels of TLR4 and RAGE were found to be increased in the colonocytes and lamina propria of patients with active ulcerative colitis. Therefore, targeting the S100A8/A9-TLR4/RAGE axis may provide therapeutic advantages for developing a broad range of anti-inflammatory and anticancer treatment methods. Therapeutic peptides targeting macrophages have several advantages, including economic benefits, feasibility of clinical-grade manufacturing, and applicability in the treatment of inflammatory diseases. By confirming the impact of peptide length and formulation on immunogenicity, the present inventors have shown that peptide-based formulations may be used and lead to groundbreaking advancements.

Abnormal activation of the nucleotide-binding oligomerization domain (NOD)-like receptor protein 3 (NLRP3) inflammasome has been found in inflamed tissues of mouse models and patients with IBD, indicating a potential pathogenic association in this disease. TLR4 and RAGE were found to be strongly associated with colonic inflammation through their involvement in the assembly of the pyrin domain (PYD), leucine-rich repeat (LRR), and NACHT domains of the NLRP3 inflammasome by the activated nuclear factor-kappa B (NF-κB) pathway. This subsequently leads to the expression of NLRP3 and interleukin-1 beta (IL-1β). In LPS-primed BMDMs, the S100A8/A9 peptide according to one embodiment of the present invention effectively inhibits the interaction between NLRP3 and apoptosis-associated speck-like protein containing a caspase recruitment domain (ASC), indicating that it is involved in regulating the activation process. The present inventors discovered that rCT-S100A8/A9 may suppress the development of colitis and CRC by regulating the activation of the NLRP3 inflammasome. In a study performed using a DSS-induced colitis model, it was found that rCT-S100A8/A9 successfully reduced the secretion of IL-1β and IL-18, which was also associated with the inhibition of NLRP3-ASC interactions. The ability of rCT-S100A8/A9 to regulate the activation of the NLRP3 inflammasome suggests its potential to disrupt the inflammatory feedback loop caused by excessive TLR4/RAGE responses and reestablish physiologically appropriate immune responses in colonic diseases.

Macrophages are increasingly recognized as critical contributors to maintaining intestinal homeostasis and as important sentinels of the intestinal immune system. Balanced differentiation and activation of macrophage subsets contributes to the overall health and function of the colonic immune system and is therefore an important factor in achieving these functions. Recently, it was reported that overexpression of S100A9 in obesity interferes with the development of M2-like macrophages through TLR4-NFκB signaling. This ultimately induces TLR4-dependent IL-1β release by S100A9 in macrophages, exacerbating skin inflammation and impairing wound healing. Indeed, S100A9 has been shown to regulate immunosuppression mediated by myeloid-derived suppressor cells (MDSCs) in colorectal cancer in a manner involving RAGE and TLR4 signaling pathways. The immunosuppressive activity of MDSC-derived macrophages depends on the persistent expression of S100A9 protein in these cells. S100A9 also promotes M2 polarization of macrophages. This highlights the role of S100A9 in regulating macrophage plasticity and functions in response to specific disease conditions and emphasizes the importance of S100A9 in shaping immune responses and inflammation associated with this disease. Considering that TLR4/RAGE expression in macrophages is highly upregulated in the inflamed colon, it is reasonable to assume that targeting S100-PRR interactions has the potential to regulate macrophage polarization toward a balanced phenotype in the context of intestinal inflammation. The present inventors further demonstrated that rCT-S100A8/A9 reduces the NLRP3 inflammasome-mediated release of IL-1β.

TLR4- and RAGE-targeted therapies are currently under development for acute and chronic inflammation. Although these inhibitors are promising, they have potential side effects, including increased susceptibility to infection and impaired wound healing. This is understandable because these PRRs play an important role in recognizing and responding to microbial pathogens. Therefore, when considering therapeutic interventions targeting TLR4 and RAGE, it is important to carefully balance the potential benefits and potential risks and develop strategies to selectively modulate rather than eliminate the activity of TLR4. From a therapeutic perspective, targeting the S100/TLR4/RAGE axis instead of directly targeting the PRR itself offers a clear advantage. By specifically blocking the interaction between S100 proteins and TLR4/RAGE, the focus is placed on impeding axis-related harmful effects while potentially preserving TLR4-dependent antibacterial defense mechanisms. Therefore, when the inflammatory feedback loop caused by excessive TLR4/RAGE responses by S100A8/A9 is disrupted, inflammation may be reduced, and physiologically appropriate immune responses to pathogens may be restored, preventing excessive production of pro-inflammatory cytokines and chemokines.

The fusion protein described herein may or may not include a signal peptide that functions to secrete the fusion protein from a host cell. A nucleic acid sequence encoding a signal peptide may be operably linked to a nucleic acid sequence encoding a protein of interest. In some embodiments, the fusion protein includes a signal peptide. In some embodiments, the fusion protein does not include a signal peptide.

In addition, the fusion protein described herein may include modified forms of protein binding peptides. For example, a fusion protein component may have post-translational modifications, including, for example, glycosylation, sialylation, acetylation, and phosphorylation, to any of the protein binding peptides.

Unless otherwise specified, the fusion protein of the invention is administered as a polypeptide (or a nucleic acid encoding a polypeptide) that is not in itself a part of a live, killed, or recombinant bacterial or viral vectored vaccine. In addition, unless otherwise specified, the fusion protein of the present invention is an isolated fusion protein, for example, not incorporated into a single parent.

In the present invention, “fusion” refers to integrating two molecules with different or identical functions or structures, and may be fusion by any physical, chemical, or biological methods capable of binding peptides. The fusion protein or a polypeptide constituting the fusion protein may be prepared by a chemical peptide synthesis method known in the art, or by amplifying a gene encoding the fusion protein by polymerase chain reaction (PCR) or synthesizing it by a known method and then cloning it into an expression vector and expressing it.

The term “polynucleotide” or “nucleic acid,” as used herein, is intended to include DNA and RNA, such as genomic DNA, cDNA, mRNA, recombinantly produced and chemically synthesized molecules. Nucleic acids may be single-stranded or double-stranded. RNA includes in vitro transcribed RNA (IVT-RNA) or synthetic RNA. According to the present invention, the polynucleotide is preferably an isolated polynucleotide.

A nucleic acid may be included in a vector. As used herein, the term “vector” includes any vector known to those skilled in the art, including a plasmid vector, a cosmid vector, a phage vector such as lambda phage, a viral vector such as a retrovirus, adenovirus or baculovirus vector, and an artificial chromosome vector such as a bacterial artificial chromosome (BAC), a yeast artificial chromosome (YAC) or a P1 artificial chromosome (PAC). The vector includes an expression vector as well as a cloning vector. An expression vector includes a viral vector as well as a plasmid vector, and generally includes an encoding sequence and a suitable DNA sequence required for expression of operably linked encoding sequences in a particular host organism (e.g., bacteria, yeast, plants, insects, or mammals) or in an in vitro expression system. Cloning vectors are generally used to engineer and amplify specific desired DNA fragments and may lack a functional sequence necessary for expressing the desired DNA fragments.

In one embodiment of all aspects of the present invention, a nucleic acid encoding the fusion protein according to the present invention is expressed in cells of an individual being treated to produce the fusion protein according to the present invention. In one embodiment of all aspects of the present invention, the nucleic acid is transiently expressed in cells of an individual. Accordingly, in one embodiment, the nucleic acid is not inserted into the genome of the cells. In one embodiment of all aspects of the present invention, the nucleic acid is RNA, preferably RNA transcribed in vitro.

The nucleic acid described herein may be a recombinant and/or isolated molecule.

The term “RNA” as used herein relates to a nucleic acid molecule including ribonucleotide residues. In a preferred embodiment, RNA encompasses all or most ribonucleotide residues. As used herein, “ribonucleotide” refers to a nucleotide having a hydroxyl group at the 2′-position of the β-D-ribofuranosyl group. RNA encompasses, but is not limited to, double-stranded RNA, single-stranded RNA, isolated RNA such as purified RNA, essentially pure RNA, synthetic RNA, recombinantly produced RNA, as well as naturally occurring RNA and modified RNA changed by addition, deletion, substitution and/or alteration of one or more nucleotides. These alterations may refer to the addition of non-nucleotide material to internal RNA nucleotides or to the RNA terminus (termini). In addition, it is contemplated that nucleotides in RNA may be chemically synthesized nucleotides or non-standard nucleotides such as deoxynucleotides. In the present invention, these altered RNAs are considered analogs of naturally occurring RNA.

In specific embodiments herein, the RNA is messenger RNA (mRNA), which is associated with an RNA transcript that encodes a peptide or protein. As established in the art, mRNA generally includes a 5′-untranslated region (5′-UTR), a peptide-encoding region, and a 3′-untranslated region (3′-UTR). In some embodiments, RNA is produced by in vitro transcription or chemical synthesis. In one embodiment, the mRNA is produced through in vitro transcription using a DNA template, and the DNA here refers to a nucleic acid containing deoxyribonucleotides.

In one embodiment, the RNA is IVT-RNA and may be obtained by in vitro transcription from a suitable DNA template. The transcriptional regulatory promoter may be any promoter for any RNA polymerase. A DNA template for in vitro transcription may be obtained by cloning a nucleic acid, especially cDNA, and introducing it into an appropriate vector for in vitro transcription. cDNA may be obtained by reverse transcription of DNA.

In one embodiment, the RNA may have modified ribonucleotides. Examples of modified ribonucleotides include, but are not limited to, 5-methylcytidine, pseudouridine, and/or 1-methyl-pseudouridine.

In some embodiments, the RNA according to the present invention includes a 5′-cap. In one embodiment, the RNA herein does not have an uncapped 5′-triphosphate. In one embodiment, RNA may be modified with a 5′-cap analog. The term “5′-cap” refers to a structure found at the 5′-terminus of an mRNA, which generally consists of a guanosine nucleotide linked to the mRNA through a 5′->5′ triphosphate linkage. In one embodiment, this guanosine is methylated at the 7-position. RNA tagged with a 5′-cap or a 5′-cap analog may be provided by in vitro transcription in which the 5′-cap is co-transcriptionally expressed into an RNA strand, or by translating it using a capping enzyme and then attaching it to the RNA.

In some embodiments, the RNA according to the present invention includes a 5′-UTR and/or 3′-UTR. The term “untranslated region” or “UTR” refers to a region in a DNA molecule that is transcribed but not translated into an amino acid sequence, or a corresponding region in an RNA molecule, such as an mRNA molecule. The UTR may be present at 5′ (upstream) of an open reading frame (5′-UTR) and/or may be present at 3′ (downstream) of the open reading frame (3′-UTR). The 5′-UTR, if present, is located at the 5′ end, upstream of the start codon of a protein-encoding region. The 5′-UTR (if present) is present downstream of the 5′-cap, for example, immediately adjacent to the 5′-cap. The 3′-UTR, if present, is located at the 3′ end, downstream of the stop codon of a protein-encoding region, but the term “3′-UTR” preferably does not include a poly(A) tail. Therefore, the 3′-UTR (if present) is present upstream of the poly(A) sequence, e.g., immediately adjacent to the poly(A) sequence.

In some embodiments, the RNA according to the present invention includes a 3′-poly(A) sequence. As used herein, the term “poly(A) sequence” or “poly-A tail” refers to a sequence of uninterrupted or interrupted adenyl (A) residues typically located at the 3′-terminus of an RNA molecule. The poly(A) sequence follows the 3′-UTR in the RNAs that are known to those skilled in the art and described herein. The poly(A) sequence may be of any length. In some embodiments, the poly(A) sequence includes or consists of at least 20, at least 30, at least 40, at least 80, or at least 100, and at most 500, at most 400, at most 300, at most 200, or at most 150 A nucleotides, and, in particular, about 110 A nucleotides.

In some embodiments, the poly(A) sequence consists only of A nucleotides. In some embodiments, as disclosed in WO 2016/005324 A1, incorporated herein by reference, the poly(A) sequence consists essentially of A nucleotides, but is interrupted by a random sequence of four types of nucleotides (A, C, G, and U). This random sequence may be 5 to 50, 10 to 30, or 10 to 20 nucleotides long. A poly(A) cassette that is present in an encoding sequence of DNA essentially consisting of dA nucleotides but interrupted by a random sequence having a length of, for example, 5 to 50 nucleotides with an equivalent distribution of the four types of nucleotides (dA, dC, dG, dT) exhibits, at the DNA level, steady propagation of plasmid DNA in E. coli, and at the RNA level, it is still associated with beneficial properties in terms of supporting RNA stability and translation efficiency.

In some embodiments, no nucleotide other than an A nucleotide is next to a poly(A) sequence at its 3′-terminus, that is, no nucleotide other than A covers or follows a poly(A) sequence at its 3′-terminus.

In the context of the present invention, the term “transcription” refers to the process in which a genetic code is transcribed from a DNA sequence into RNA. Thereafter, the RNA may be translated into a peptide or protein.

“Encoding” refers to the inherent property of a specific sequence of nucleotides in a polynucleotide, such as a gene, cDNA, or mRNA, that has a defined sequence of nucleotides (i.e., rRNA, tRNA, and mRNA) or a defined sequence of amino acids and biological properties derived therefrom and that allows the sequence to serve as a template for synthesis of other polymers and macromolecules in biological processes. Therefore, when transcription and translation of an mRNA corresponding to this gene produce a protein in a cell or other biological system, the gene encodes a protein. Both a coding strand, which is identical to an mRNA sequence and is generally provided in a sequence listing, and a non-coding strand, which is used as a template for transcription of a gene or cDNA, may be mentioned as encoding a protein or another product of this gene or cDNA.

As used herein, “endogenous” refers to any substance derived or produced within an organism, cell, tissue, or system.

As used herein, “exogenous” refers to any substance derived or produced outside of an organism, cell, tissue, or system.

As used herein, the term “expression” is defined as the transcription and/or translation of a specific nucleotide sequence.

As used herein, the terms “linked” and “fused” or “fusion” are used interchangeably. These terms mean two or more elements or components or domains are linked together.

The peptides, proteins, polypeptides, RNA, RNA particles and additional substances described herein, such as the rCT-S100A8/A9 fusion protein, may be administered as a pharmaceutical composition or medicament for therapeutic or prophylactic treatment, and may be administered in the form of any appropriate pharmaceutical composition that may include a pharmaceutically acceptable carrier or that may optionally include one or more adjuvants, stabilizers, and the like. In one embodiment, the pharmaceutical composition is for use in therapeutic or prophylactic treatment, for example, in treating or preventing an antigen-associated disease, such as a cancer disease, for example, those described herein.

The term “pharmaceutical composition” relates to a formulation including a therapeutically effective substance, preferably together with pharmaceutically acceptable carriers, diluents, and/or excipients. The pharmaceutical composition is useful for reducing the severity of, preventing, or treating a disease or disorder by administering the pharmaceutical composition to an individual. A pharmaceutical composition is also known in the art as a pharmaceutical formulation. In the context of the present invention, pharmaceutical compositions include peptides, proteins, polypeptides, RNA, RNA particles, immune effector cells, and/or additional substances as described herein.

The pharmaceutical composition of the present invention may include one or more adjuvants or may be administered together with one or more adjuvants. The term “adjuvant” refers to a compound that prolongs, enhances, or accelerates an immune response. Adjuvants include a heterogeneous group of compounds such as oil emulsions (e.g., Freund's adjuvant), mineral compounds (e.g., alum), bacterial products (e.g., pertussis toxin) or immune stimulating complexes. Examples of adjuvants include, but are not limited to, LPS, glycoprotein 96 (GP96), cytosine-phosphate-guanine (CpG) oligodeoxynucleotides, growth factors, and cytokines such as monokines, lymphokines, interleukins, and chemokines. The cytokine may be IL1, IL2, IL3, IL4, IL5, IL6, IL7, IL8, IL9, IL10, IL12, IFNα, IFNγ, granulocyte-macrophage colony-stimulating factor (GM-CSF), and lymphotoxin-alpha (LT-α). Additional known adjuvants are aluminum hydroxide, Freund's adjuvant, or oils such as MONTANIDE® ISA51. Other adjuvants suitable for use herein include lipopeptides such as Pam3Cys.

The pharmaceutical composition according to the present invention is generally applied in a “pharmaceutically effective amount” and as a “pharmaceutically acceptable formulation.”

The term “pharmaceutically acceptable” refers to the non-toxic nature of a substance that does not interact with the action of an active ingredient of a pharmaceutical composition.

The term “pharmaceutically effective amount” or “therapeutically effective amount” refers to the amount that, alone or in combination with additional administration, achieves the desired response or desired effect. When treating a particular disease, the desired response preferably refers to inhibition of the progression of the disease. This includes slowing down the progression of the disease, and in particular, stopping or reversing the progression of the disease. The desired response in the treatment of a disease may also be delaying the onset or preventing the onset of the disease or condition. The effective dose of the composition described herein will be determined depending on the condition being treated, the severity of the disease, the individual characteristics of the patient, including age, physical condition, height, and weight, the duration of treatment, the type of concomitant therapy (if any), the specific route of administration, and similar factors. Therefore, the administered dosage of the composition described herein may be determined according to these various properties. When the patient does not sufficiently respond to the first dose, higher doses (or effectively, higher doses achieved by other, more local routes of administration) may be used.

The pharmaceutical composition of the present invention may include salts, buffers, preservatives, and optionally other therapeutic agents. In one embodiment, the pharmaceutical composition of the present invention include one or more pharmaceutically acceptable carriers, diluents, and/or excipients.

Preservatives suitable for use in the pharmaceutical composition of the present invention include, but are not limited to, benzalkonium chloride, chlorobutanol, parabens, and thimerosal.

The term “excipient” as used herein refers to a substance that may be present in the pharmaceutical composition of the present invention but is not an active ingredient. Examples of excipients include, but are not limited to, carriers, binders, diluents, lubricants, thickeners, surfactants, preservatives, stabilizers, emulsifiers, buffers, flavoring agents, or colorants.

The term “diluent” refers to a diluting and/or thinning agent. In addition, the term “diluent” includes any one or more of fluids, liquids or solid suspensions, and/or mixed media. Examples of suitable diluents include ethanol, glycerol, and water.

The term “carrier” refers to an ingredient, which may be natural, synthetic, organic, or inorganic, that is combined with an active ingredient to facilitate, enhance, or allow administration of the pharmaceutical composition. As used herein, a carrier may be one or more compatible solid or liquid fillers, diluents, or encapsulating materials suitable for administration to an individual. Suitable carriers include, but are not limited to, sterile water, Ringer's solution, Ringer's lactate, sterile sodium chloride solution, isotonic saline, polyalkylene glycols, hydrogenated naphthalenes, and, especially, biocompatible lactide polymers, lactide/glycolide copolymers, or polyoxyethylene/polyoxypropylene copolymers. In one embodiment, the pharmaceutical composition of the present invention includes isotonic saline.

Pharmaceutically acceptable carriers, excipients, or diluents for therapeutic use are well known in the pharmaceutical field and are described, for example, in Remington's Pharmaceutical Sciences, Mack Publishing Co. (A. R Gennaro edit. 1985).

Pharmaceutical carriers, excipients, or diluents may be selected depending on the intended route of administration and standard pharmaceutical practices.

In one embodiment, the pharmaceutical composition described herein may be administered intravenously, intraarterially, subcutaneously, intradermally, or intramuscularly. In specific embodiments, the pharmaceutical composition is formulated for topical or systemic administration. Systemic administration may include enteral administration involving absorption through the gastrointestinal tract or parenteral administration. As used herein, “parenteral administration” refers to administration by any method other than through the gastrointestinal tract, such as by intravenous injection. In a preferred embodiment, the pharmaceutical composition is formulated for systemic administration. In another preferred embodiment, systemic administration is by intravenous administration. In one embodiment of all aspects of the invention, the RNA encoding the rCT-S100A8/A9 fusion protein described herein and optionally the RNA encoding an antigen are administered systemically.

The term “co-administration” as used herein refers to the administration of several compounds or compositions (e.g., immune effector cells, RNA encoding an IL2 variant polypeptide, and optionally RNA encoding a vaccine antigen) to the same patient. Several compounds or compositions may be administered simultaneously, essentially simultaneously, or sequentially.

The materials, compositions, and methods described herein may be used to treat individuals with a disease, for example, a disease characterized by the presence of diseased cells that express inflammation. Particularly preferred diseases are colitis, colorectal cancer, colon inflammation, or colon cancer.

The materials, compositions, and methods described herein may be used in the therapeutic or prophylactic treatment of a variety of diseases, and here, as described herein, the support of and/or the activation of immune effector cells is advantageous for patients with inflammatory diseases or cancer.

The term “disease” refers to an abnormal condition affecting an individual's body. A disease is often interpreted as a medical condition associated with specific symptoms and signs. A disease may be caused by factors originating from an external source such as an infectious disease, or may be caused by an internal dysfunction such as an autoimmune disease. In humans, “disease” is used in a broader sense to refer to any condition that, upon contact with an individual, causes pain, dysfunction, distress, social problems, death, or similar problems in the afflicted individual. In a broader sense, it sometimes includes injuries, disabilities, disorders, syndromes, infections, isolated symptoms, deviant behavior, and atypical variants, both structural and peptide combinations, and which in other contexts and for other purposes, are considered distinct categories. A disease generally affects an individual not only physically but also emotionally, as living with various diseases may change an individual's perspective on life and personality.

In this context, the terms “treatment,” “treating,” or “therapeutic intervention” refer to the management and care of an individual for the purpose of combating a condition such as a disease or disorder. The terms are intended to include not only ameliorating symptoms or complications, delaying the progression of a disease, disorder, or condition, ameliorating or alleviating symptoms and complications, and/or curing or eliminating a disease, disorder, or condition, but also the full-range treatment for a given condition from which an individual suffers, such as the administration of a therapeutically effective compound to prevent a condition, and here, prevention will be understood as the management and care of an individual for the purpose of combating a disease, condition, or disorder, and includes the administration of an active compound to prevent the onset of symptoms or complications.

The term “therapeutic treatment” refers to any treatment that improves the health status of an individual and/or prolongs (increases) the lifespan of the individual. The treatment may be eliminating a disease in an individual, stopping or slowing down the progression of a disease in an individual, inhibiting or slowing down the progression of a disease in an individual, reducing the frequency or severity of symptoms in an individual, and/or reducing the reoccurrence of a disease in an individual who is currently suffering from the disease or has previously suffered from the disease.

The term “prophylactic treatment” or “preventive treatment” refers to any treatment intended to prevent a disease from developing in an individual. The term “prophylactic treatment” or “preventive treatment” are used interchangeably herein.

Improvement may be indicated by an improvement in a disease activity index, by amelioration of clinical symptoms, or by any other measure of disease activity. One such disease index is the Mayo Score for Ulcerative Colitis. The Mayo Score is an established, validated disease activity index for mild, moderate, and severe ulcerative colitis (UC) calculated as the sum of four sub-scores of stool frequency, rectal bleeding, endoscopy, Physician's Global Assessment (PGA), and it ranges from 0 to 12. A score of 3 to 5 points indicates mildly active disease, a score of 6 to 10 points indicates moderately active disease, and a score of 11 to 12 points indicates severe disease. The partial Mayo score, which is the Mayo score without the endoscopy sub-score, is calculated as the sum of the stool frequency, rectal bleeding, and PGA sub-scores and ranges from 0 to 9. The modified Mayo score, which is the Mayo score without the PGA sub-score, is calculated as the sum of the stool frequency, rectal bleeding, and endoscopy sub-scores and ranges from 0 to 9. Other disease activity indices for UC include, for example, the Ulcerative Colitis Endoscopic Index of Severity (UCEIS) score and the Bristol Stool Form Scale (BSFS) score. The UCEIS score provides an overall assessment of the endoscopic severity of UC based on mucosal vascular patterns, bleeding, and ulceration (Travis et al., Gut. 61:535-542(2012)). The score ranges from 3 to 11, and a higher score indicates more severe disease based on endoscopy. The BSFS score is used to classify the forms (or mass) of human excrement into seven categories (Lewis and Heaton, Scand J Gastroenterol. 32(9):920-924 (1997)).

The terms “subject” and “individual” are used interchangeably herein. These terms refer to humans or other mammals (e.g., mice, rats, rabbits, dogs, cats, cows, pigs, sheep, horses, or primates) that may suffer from or be susceptible to a disease or disorder (e.g., cancer), but they may or may not suffer from the disease or disorder. In many embodiments, the individual is a human. Unless otherwise specified, the terms “subject” and “individual” do not refer to a specific age and therefore encompass adults, the elderly, children, and newborns. In embodiments of the present invention, an “individual” or “subject” is a “patient.”

The term “patient” refers to a subject or individual in need of treatment, specifically a subject or individual suffering from a disease.

In one embodiment of the present invention, the goal is to treat a disease by inhibiting the expression of inflammatory cytokines in inflammatory cells expressing inflammation, increasing the survival rate of individuals with inflammatory cells, killing cancer cells, or inhibiting the proliferation of cancer cells, or increasing the survival rate of individuals bearing cancer cells.

### EXAMPLES

### Example 1. Materials and Methods

### 1.1 Mice and Cell Culture

Wild-type C57BL/6 mice were purchased from Samtako Bio Korea (Gyeonggi-do, Korea). S100a8-KO (C57BL/6Smoc-S100a8em1Smoc) and S100a9-KO (C57BL/6Smoc-S100a9em1Smoc) mice were purchased from Shanghai Model Organisms Center Inc. (Jinshan, PR China). Primary bone marrow-derived macrophages (BMDMs) were isolated from mice and cultured in Dulbecco's modified eagle's medium (DMEM) in the presence of macrophage colony-stimulating factor (M-CSF) (416-ML; R&D Systems Inc.) for three to five days. HEK293T (ATCC-11268; American Type Culture Collection) and human monocyte THP-1 (ATCC-TIB-202) cells were cultured in DMEM (GenDEPOT) and RPMI (GenDEPOT) containing 10% fetal bovine serum (FBS; Gibco Inc.), sodium pyruvate, non-essential amino acids, and penicillin.

To establish a CRC cell model resistant to oxaliplatin, doxorubicin, or 5-FU, HT29, HT116, and SW480 cells were exposed to progressively increasing concentrations of oxaliplatin, doxorubicin, or 5-FU. The established oxaliplatin, doxorubicin, and 5-FU resistant cells were named OxaR, DoxR, and 5-FuR cells, respectively.

### 1.2 Reagents and Antibodies

LPS (Escherichia coli O111:B4, tlrl-eblps), adenosine 5′-triphosphate (ATP, tlrl-atpl), nigericin, monosodium urate crystals (MSU, tlrl-msu), poly(dA:dT) (tlrl-patc), and flagellin (FLA-ST, tlrl-stfla) were purchased from Invivogen. DSS (36 to 50 kDa) was purchased from MP Biomedicals.

An antibody specific for nicotinamide phosphoribosyltransferase (NAMPT; ab236874) was purchased from Abcam. RAGE (RD9C2), TLR4 (25), actin (I-19), ASC (N-15-R), IL-18 (H-173-Y), caspase-1 p10 (M-20), HA (12CA5), Flag (D-8), GST (B-14), Myc (9E10), and His (H-3) were purchased from Santa Cruz Biotechnology. Specific antibodies against S100A8 (E4F8V), S100A9 (D3U8M), E-cadherin (24E10), N-cadherin (D4R1H) and β-catenin (D10A8) were purchased from Cell Signaling Technology (Danvers, MA, USA). IL-1β (AF-401-NA) and NLRP3 (AG-20B-0014) were purchased from R&D Systems and Adipogen, respectively.

### 1.3. Plasmid Construction

HA or His-S100A8 or S100A9, Myc-TLR4 and Myc-RAGE plasmids were purchased from Addgene. Plasmids encoding various regions of S100A8 or S100A9 were prepared by PCR amplification from full-length S100A8 or S100A9 cDNA and subcloning into a pEBG derivative encoding an N-terminal glutathione S-transferase (GST) epitope tag between BamHI and NotI sites. All constructs were sequenced to confirm 100% identity with the original sequence. The mRNA sequences of S100A8 and S100A9 are accession no. NM_013650 and NM_001281852, respectively, and the protein sequences are accession no. NP_038678, and NP_001268781, respectively.

### 1.4 Peptides

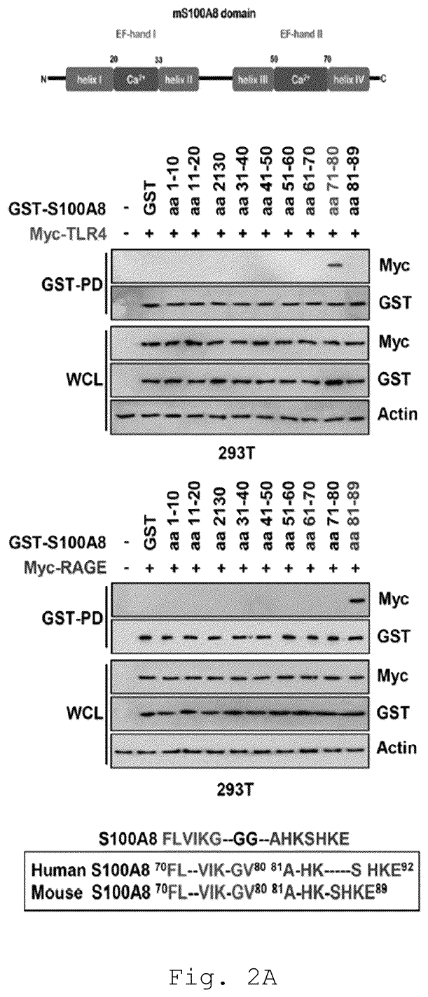

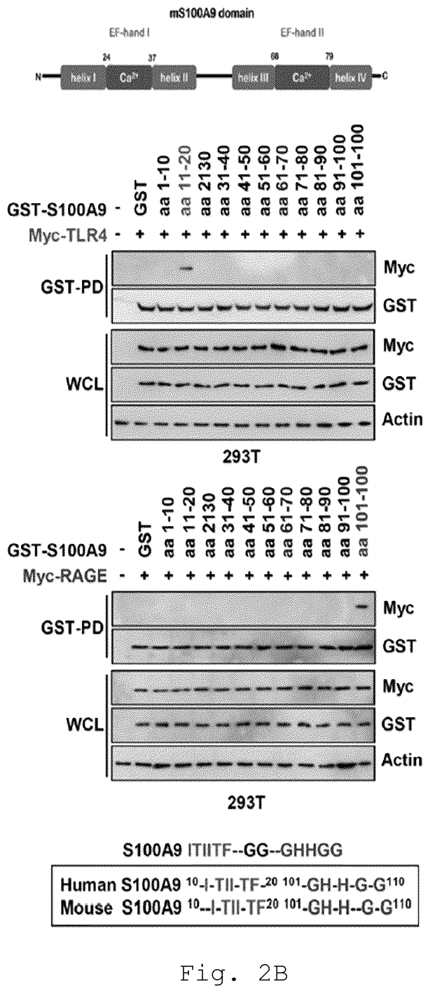

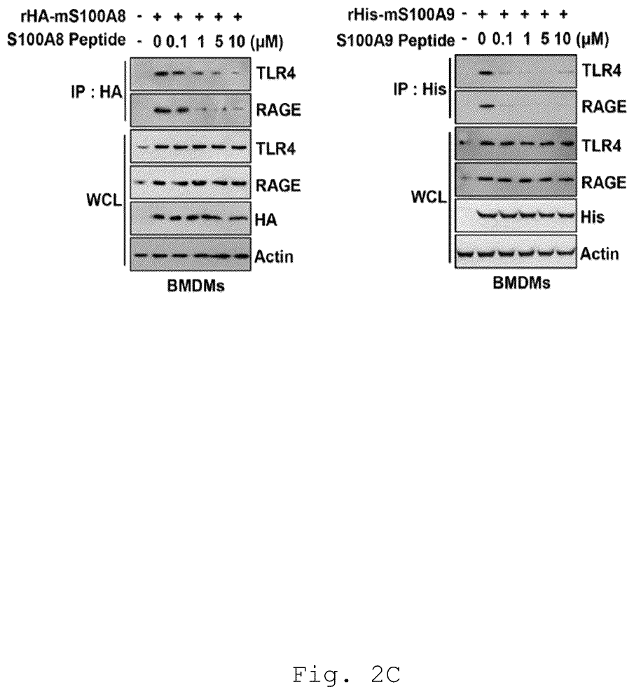

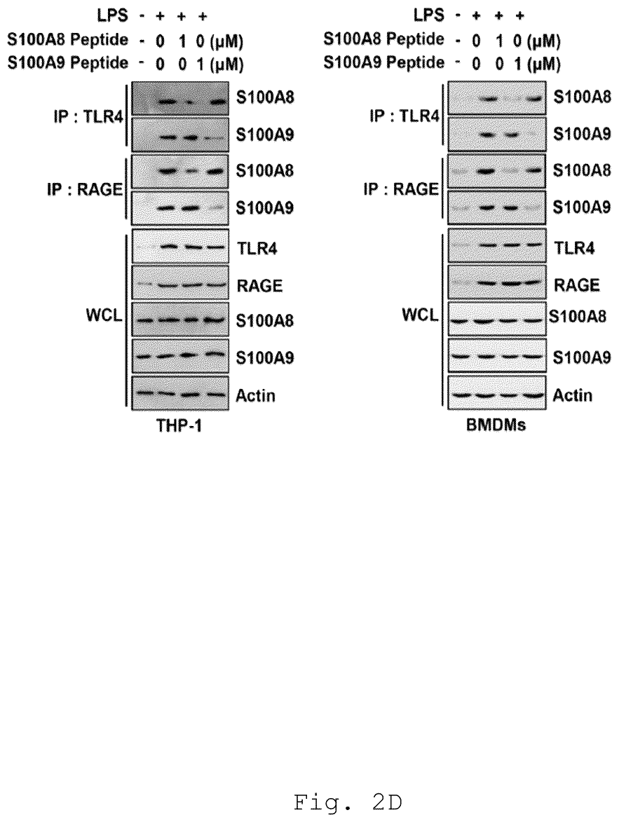

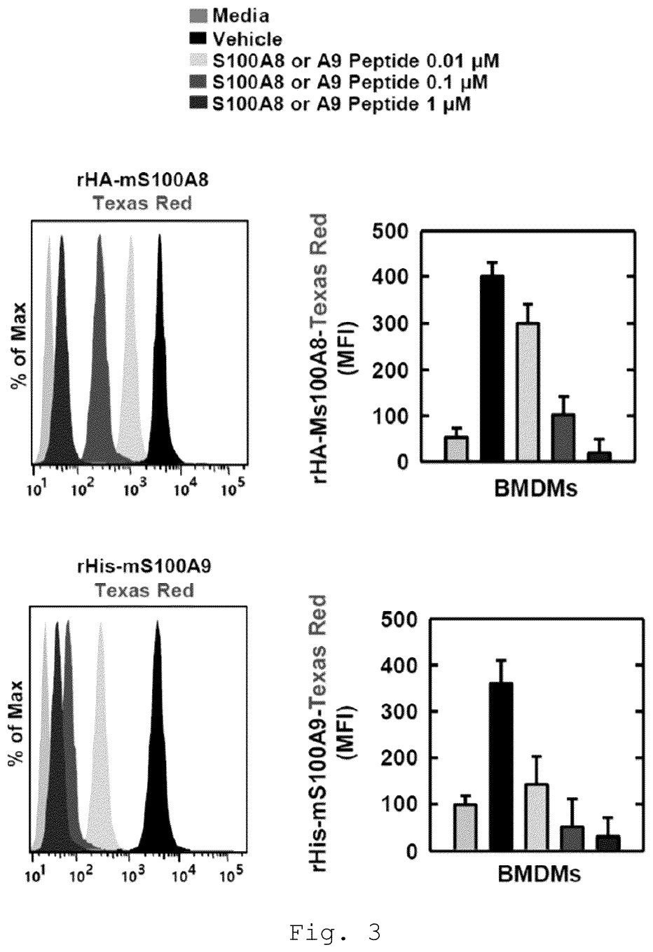

S100A8 or S100A9 peptides were commercially synthesized and purified in the form of an acetate salt by Peptron (Korea) to avoid abnormal intracellular reactions. The amino acid sequences of the peptides in the present study are shown in FIGS. 2A, 2B, and 3B. The endotoxin content measured by Limulus amebocyte lysate assay (BioWhittaker) was less than 3 to 5 pg/ml at the peptide concentrations used in the experiment.

### 1.5 Recombinant Proteins

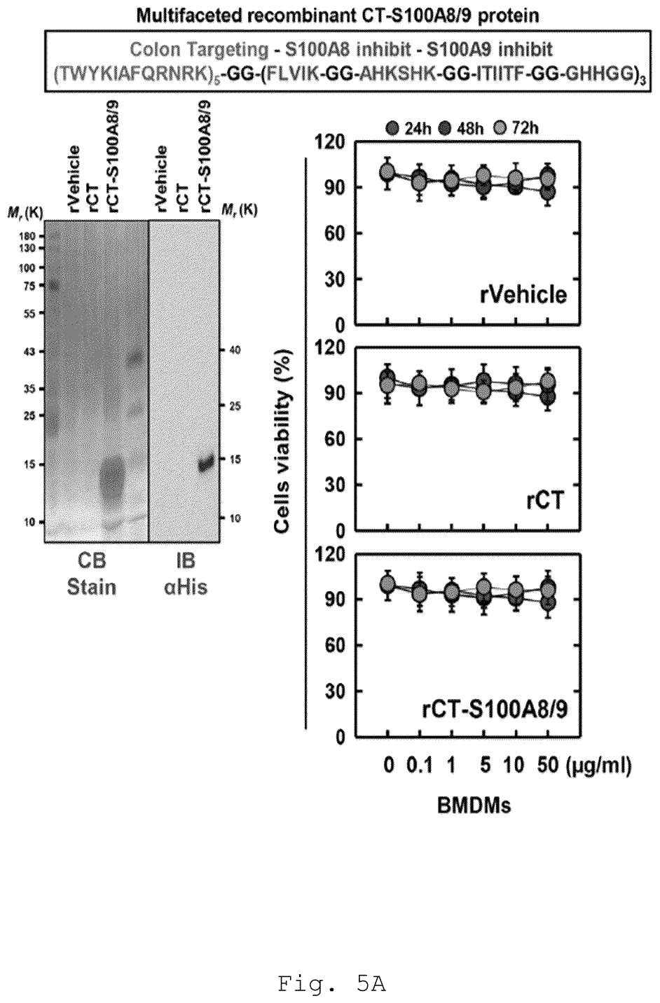

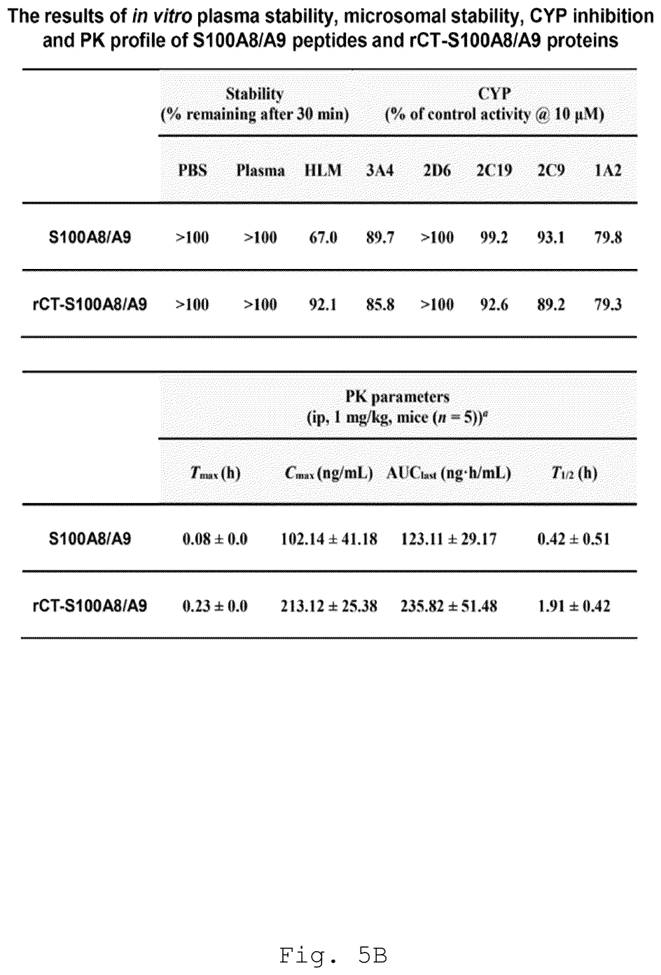

To obtain the recombinant rCT-S100A8/9 protein (FIG. 5A), the S100A8 or S100A9 amino acids (FIGS. 2A, 2B, and 3B) and the CT peptide (TWYKIAFQRNRK) colon targeting sequence were cloned into the pRSFDuet-1 vector (Novagen) using an N-terminal 6×His tag. Then, expression induction, harvesting, and purification were performed from the Escherichia coli expression strain BL21(DE3) pLysS according to the standard protocol recommended by Novagen. rVehicle, rCT or rCT-S100A8/9 were dialyzed through permeable cellulose membranes and tested for LPS contamination via Limulus amebocyte lysate assay (BioWhittaker) and found to contain <20 pg/ml LPS.

### 1.6 GST Pulldown, Immunoblot (IB), and Immunoprecipitation (IP) Analyses

293T, BMDMs, and THP-1 cells were treated as indicated and processed for analysis by GST pulldown, Western blotting, and co-IP analysis. For GST pulldown, 293T cells were harvested and lysed in an NP-40 buffer including a complete protease inhibitor cocktail (Roche, Basel, Switzerland). After centrifugation, the supernatant was pre-cleared using protein A/G beads for two hours at 4° C. The pre-cleared lysates were mixed with a 50% slurry of glutathione-conjugated Sepharose beads (Amersham Biosciences, Amersham, UK), and binding reactions were performed for four hours at 4° C. by incubating the lysates at 4° C. The precipitate was extensively washed with a lysis buffer. Proteins bound to the glutathione beads were eluted by boiling for five minutes in a sodium dodecyl sulfate (SDS) loading buffer. For IP, cells were harvested and lysed in an NP-40 buffer supplemented with a complete protease inhibitor cocktail (Roche, Switzerland). After pre-clearing with protein A/G agarose beads for one hour at 4° C., whole cell lysates were used for IP using the indicated antibodies. Typically, 1 to 4 g of a commercial antibody was added to 1 ml of the cell lysate, and the resulting mixture was incubated for 8 to 12 hours at 4° C. After adding protein A/G agarose beads for six hours, immunoprecipitates were extensively washed with a lysis buffer and eluted with an SDS loading buffer by boiling for five minutes. For immunoblotting (IB), polypeptides were analyzed by SDS-polyacrylamide gel electrophoresis and transferred to polyvinylidene difluoride (PVDF) membranes (Bio-Rad, Hercules, CA, USA). Immunodetection was performed using specific antibodies. Antibody binding was visualized by chemiluminescence (ECL; Millipore, Burlington, MA, USA) and detected using a Vilber chemiluminescence analyzer (Fusion SL 3; Vilber Lourmat).

### 1.7 Flow Cytometry

S100A8 or S100A9 peptides or rCT-S100A8/9 was conjugated to Texas Red using the Texas Red® Conjugation Kit (Fast)-Lightning-Link (Abcam) to quantitatively measure cellular uptake in macrophages and pharmacodynamics in a colitis model. Cells were washed thoroughly and quickly with pulse spins and immediately acquired for analysis on FACSCalibur (BD Biosciences, San Jose, CA, USA). Data was plotted using CellQuest software (BD Biosciences) and analyzed using FlowJo software (Tree Star, Ashland, OR, USA).

### 1.8 Enzyme-Linked Immunosorbent Assay (ELISA)

Cytokine content was analyzed in cell culture supernatants and mouse sera using the BD OptEIA ELISA set (BD Pharmingen) to detect TNF-α, IL-6, IL-1β, and IL-18. All analyses were performed according to the manufacturer's recommendations.

### 1.9 Mouse Models

DSS-induced acute or chronic colitis and AOM/DSS CRC mouse models were prepared using 6-week-old C57BL/6 female mice (Samtako Bio, Osan, Korea). To assess the induction of acute colitis, mice were exposed ad libitum to 3% (w/v) dextran sodium sulfate (molecular weight: 36-50 kDa, MP Biomedicals, Santa Ana, CA, USA) dissolved in drinking water. The mouse models are illustrated in FIGS. 7A, 5E, and 6A. DSS solutions were prepared every two days. Control mice that did not consume DSS were administered sterile distilled water. The humane endpoint for weight loss (requiring euthanasia) was 20% (compared to the original body weight of an animal). Without an approved exception request, body weight loss could not exceed 20%.

For tumor xenograft experiments, 4- to 6-week-old female athymic nude mice (Central Lab Animal Inc., Seoul, Korea) were used. 1×10⁶ HCT116 cells suspended in 0.1 mL of a cell culture medium were injected subcutaneously into the animals' right axillary region and observed for 7 to 10 days while the tumor volume was measured. Treatment was initiated when the tumor size reached an average volume of 200 or 600 mm³. The tumor volume was measured with a skin caliper every three days, calculated as (tumor length)×(tumor width)²×0.5, and expressed in mm³. All animals were maintained in a specific pathogen-free environment. All animal-related procedures were examined and approved by the Hanyang University Institutional Animal Care and Use Committee (protocol numbers: 2022-0145 and 2022-0266).

### 1.10 Clinical Score and Histology

The clinical score of colitis was obtained by measuring body weight, fecal occult blood or total blood loss, and stool consistency every other day during the induction of colitis. The clinical score were assessed by two trained investigators who were randomly selected. For immunohistochemical analysis of tissue sections, mouse distal colon tissues were fixed in 10% formalin and embedded in paraffin. Paraffin sections (4 m) were cut and stained with H&E. Histopathological scoring was performed by a certified pathologist (Min-Kyung Kim, Department of Pathology, Kim Min-Kyung Pathology Clinic, Seoul, Korea) who scored each organ section independently without prior knowledge of the treatment group.

### 1.11 CYP450 Analysis

CYP inhibition, microsomal stability, and plasma stability experiments were all performed by Drug Development & Optimization of the Osong Medical Innovation Foundation (OMIF) in Korea. Human liver microsomes (0.25 mg/mL), 0.1 M phosphate buffer (pH 7.4), and a cocktail of five probe substrates (phenacetin 50 μM, diclofenac 10 μM, S-mephenytoin 100 μM, dextromethorphan 5 μM, and midazolam 2.5 μM). S100A8/A9 peptide was added at concentrations of 0 μM (control) and 10 μM. rCT-S100A8/A9 protein was added at concentrations of 0 μg/mL (control) and 10 μg/mL. After incubation at 37° C. for five minutes, a nicotinamide adenine dinucleotide phosphate (NADPH)-generating system solution was also added and incubated again at 37° C. for 15 minutes. To terminate the reaction, acetonitrile containing an internal standard (terfenadine) was added, and the resulting mixture was centrifuged for five minutes (14,000 rpm, 4° C.). The supernatant was then injected into a liquid chromatography-mass spectrometry/mass spectrometry (LC-MS/MS) system to simultaneously analyze the metabolites of the probe substrate and evaluate the CYP inhibition rate (%) of the tested substances.

### 1.12 Microsome Stability Analysis

0.1 M phosphate buffer (pH 7.4) and S100A8/A9 peptide (10 PM) or rCT-S100A8/A9 protein (10 μg/mL) were added to human liver microsomes (0.5 mg/mL). After incubation at 37° C. for five minutes, the NADPH-generating system solution was also added and incubated again at 37° C. for 30 minutes. To terminate the reaction, acetonitrile containing an internal standard (clopramide) was added, and the resulting mixture was centrifuged for five minutes (14,000 rpm, 4° C.). The supernatant was then injected into an LC-MS/MS system to analyze the microsomal stability of the tested substances.

### 1.13 Plasma Stability Analysis

Human plasma of each culture tube treated with S100A8/A9 peptide (10 μM) or rCT-S100A8/A9 protein (10 μg/mL) was incubated at 37° C. for 0, 30, and 120 minutes, respectively. At the designated time, an acetonitrile internal standard solution of chlorpropamide was added to each culture tube, and the resulting mixture was shaken with a vortex mixer for five minutes and centrifuged for additional five minutes (14,000 rpm, 4° C.). The supernatant was then injected into an LC-MS/MS system to analyze the plasma stability of the tested substances.

### 1.14 PK Study

Female C57BL/6 mice with body weight of 16 to 18 g were used for PK studies. S100A8/A9 peptide or rCT-S100A8/A9 protein (1 mg/kg) was administered to the mice by intraperitoneal injection. Whole blood samples (40 L) were collected serially from the orbital vein at 0.17, 0.5, 1, 2, 4, and 6 hours after drug administration. Blood samples were centrifuged at 12,000 rpm for five minutes at 4° C., and plasma was collected. To a 10 μL plasma sample, 20 μL of 0.1% formic acid in acetonitrile was added along with amlodipine, which was an internal standard (IS), at a concentration of 500 ng/mL, and the resulting mixture was vortexed. Samples were then centrifuged at 13,200 rpm for five minutes, and 10 μL aliquots were injected for LC-MS/MS analysis.

### 1.15 RT² Profiler PCR Array Test

Total RNA was extracted from tissues using TRIzol reagent (Thermo Fisher Scientific, USA) according to the manufacturer's instructions. RNA quality was confirmed using a spectrophotometer, and the RNA quality control parameter OD260/280 was between 1.8 and 2.0. Reverse transcription was performed using the All-in-One™ First-Strand cDNA Synthesis kit (GeneCopoeia Inc., USA) under the following reaction conditions: 42° C. for 60 minutes and 70° C. for 5 minutes. cDNA was used in real-time RT² Profiler PCR Array (QIAGEN N.V., Cat. no. PARN-090Z) with RT2 SYBR®Green qPCR Master Mix (Cat. no. 330,529). Each array plate included one set of 96 wells for testing. gDNA contamination, reverse transcription, and positive PCR controls were included in each 96-well set of each plate. Glyceraldehyde-3-phosphate-dehydrogenase (GAPDH) was used as a reference gene for analysis. CT values were derived as an Excel file, a CT value table was created, and then uploaded to the data analysis web portal (http://www.qiagen.com/geneglobe). The samples included controls and test groups, and the CT values were normalized.

### 1.16. Statistical Analysis

All data was analyzed using Student's t-test with Bonferroni correction or analysis of variance (ANOVA) for multiple comparisons and expressed as mean±standard deviation (SD). Statistical analysis was performed using the SPSS (Version 12.0) statistical software program (SPSS Inc., Chicago, IL, USA). Differences are considered significant at p<0.05. For the survival rate, data was plotted for visual representation and analyzed by the Kaplan-Meier product limit method using the log-rank (Mantel-Cox) test for comparisons using GraphPad Prism (version 5.0; La Jolla, CA, USA).

### 2. Results

2.1 Interaction Between S100A8 or A9 and TLR4 or RAGE is Increased in Colon Lysates of Mice with Acute Colitis, Chronic Colitis and CRC.

Under inflammatory stimulation, S100A8 and S100A9 are secreted at local sites of inflammation and interact with TLR4 or RAGE of immune cells to amplify the inflammatory signaling cascades. To determine whether the S100 proteins physically interact with TLR4 or RAGE, co-IP experiments were performed under two conditions. Each of the plasmids with a designated tag (Myc-TLR4 or RAGE, HA-S100A8, and His-S100A9) was expressed in HEK293T cells or co-immunoprecipitated with each cell lysate sample after treatment with recombinant S100A8 or S100A9 proteins (rHA-mS100A8 or rHis-mS100A9) in BMDMs.

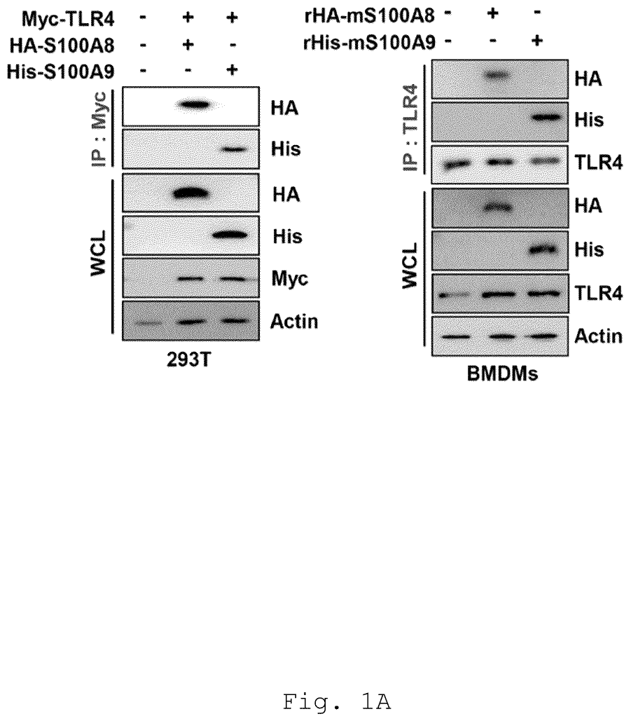

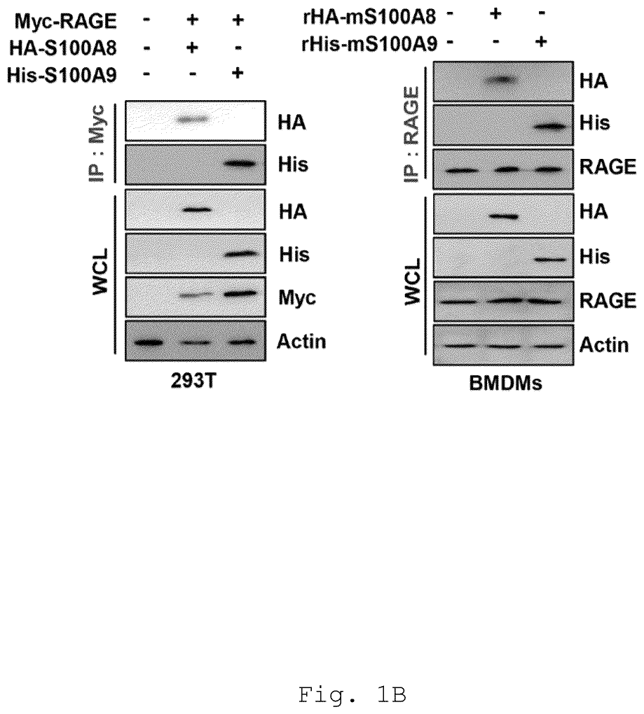

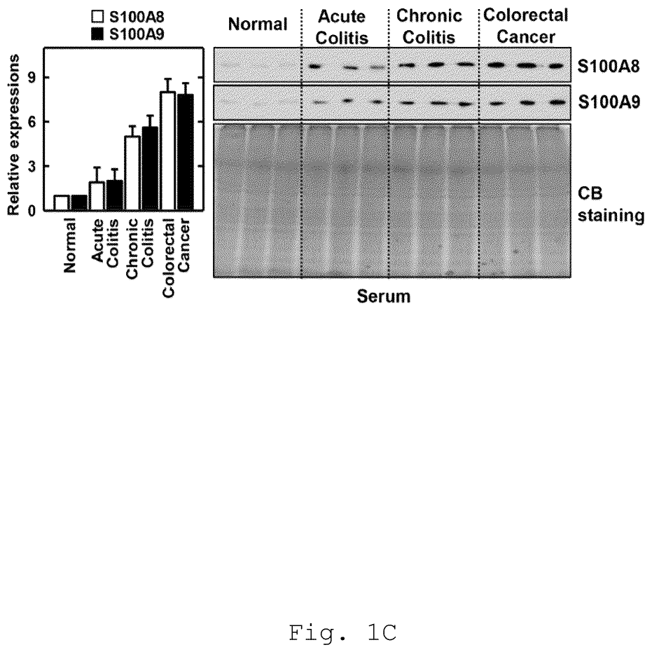

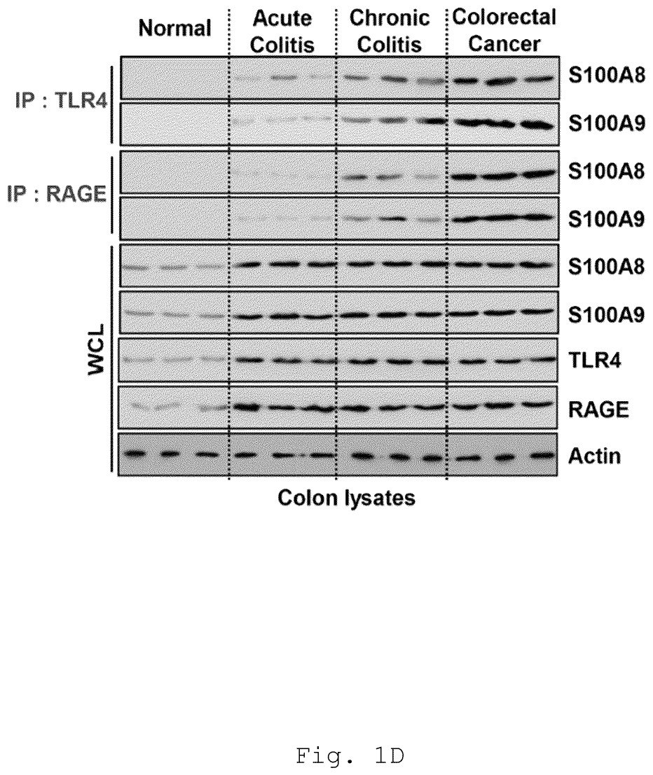

In HEK293T, the co-IP analysis demonstrated that HA-S100A8 and His-S100A9 physically interact with Myc-TLR4 or Myc-RAGE. In BMDMs, the co-IP analysis demonstrated that both rS100A8 and A9 physically interacted with endogenous TLR4 or RAGE, and interestingly, rHis-mS100A9 exhibited stronger binding to PRRs (TLR4 and RAGE) than rHA-mS100A8 (FIGS. 1A and 1B).

The physical interaction between S100 and PRR is related to its role in inflammatory responses, i.e., a peptide bond. Therefore, the present inventors hypothesized that abnormal S100 expression may be associated with colonic diseases such as IBD and CRC. To evaluate whether S100A8 and A9 are risk factors for IBD and CRC, DSS-induced colitis models, including mouse models of acute colitis, chronic colitis, and CRC, were produced. The present inventors further analyzed S100A8 and A9 expression and their physical interactions with PRRs in the sera and colonic lysates of the acute and chronic colitis and CRC mouse models. The serum S100A8 and A9 protein levels significantly increased in the order of acute colitis<chronic colitis<colon cancer mouse models. This suggests that the secretion of S100A8 and A9 increases in association with the inflammation level, and it was found that the secretion was higher in CRC than in the inflammatory diseases (FIG. 1C). In addition, physical interactions between S100A8 or A9 and TLR4 or RAGE were observed in colonic lysates of each model, which was prominent in the chronic colitis and CRC models, and the intracellular expression levels of S100A8 and A9 were observed to be similar between these models. Consistent with these findings, the physical interactions between S100A8 or A9 and TLR4 or RAGE were significantly upregulated in the mouse models of chronic colitis and CRC compared to the levels in the acute colitis model (FIG. 1D).

In line with these findings, the physical interactions of S100A8 and A9 with PRRs was noticeably increased in the colonic lysates of the acute colitis, chronic colitis, and CRC mouse models in association with increased disease severity. In summary, the results suggest that extracellular binding of S100A8 and S100A9 proteins to PRRs may increase due to chronic inflammation in the colon, thereby contributing to the development of CRC. The presence of chronic inflammation caused by the S100 proteins exacerbated disease progression, indicating a link between inflammation and cancer. Moreover, this supports the association between high serum levels of S100A8/S100A9 and clinical characteristics of patients with CRC.

### 2.2 S100A8/A9-Derived Peptides Effectively Block the Interaction Between TLR4 and RAGE.

Both S100A8 and A9 include two calcium-binding EF-hands (EF-hands I and II), with helix-loop-helix motifs linked by a hinge region and flanked by N- and C-terminal domains. To identify the amino acid (aa) residues of each S100 protein that interact with TLR4 and RAGE, vectors expressing GST-tagged truncated mutant S100A8 or A9 were constructed. Each truncated variant was designed by dividing the S100 protein sequence into 10-aa segments, starting from the N-terminus. Then, each truncated variant was detected by a GST pull-down assay in the presence of Myc-TLR4 or RAGE and subjected to IB analysis. Domain mapping showed that TLR4 interacts with S100A8 (aa 71-80) and S100A9 (aa 11-20), whereas RAGE is associated with S100A8 (aa 81-89) and S100A9 (aa 101-110) (FIGS. 2A and 2B).

Using these potential TLR4- or RAGE-interacting motifs, “S100A8 and S100A9 peptides” consisting of the TLR4- or RAGE-interacting motifs of the S100A8 and S100A9 sequences were then synthesized. Here, the amino acid sequences of the motifs are amino acids shared between mouse S100A8 and human S100A8 and between mouse S100A9 and human S100A9 to adjust the sequence differences between humans and mice (FIGS. 2A and 2B, bottom).

Considering the possibility of the S100A8 (or S100A9) peptide mimicking the common binding site and rHA-mS100A8 (or rHis-mS100A9) interactions with TLR4 or RAGE, the present inventors hypothesized that r-mS100A8 (or r-mS100A9) and S100A8 (or S100A9) peptides may compete for binding with TLR4 or RAGE. To test this hypothesis, an in vitro binding assay was performed using Texas Red-labeled rS100A8 or A9 and unlabeled A8 or A9 peptides in BMDMs by flow cytometry. To monitor the decrease in fluorescence due to binding to TLR4 or RAGE, binding assays were performed with increasing concentrations of the A8 or A9 peptide in the presence of Texas Red-labeled rS100A8 or A9. The results showed that target BMDMs showed a clear decrease in fluorescence intensity with increasing concentrations of the A8 or A9 peptide (FIG. 3). Interestingly, the S100A9 peptide showed a stronger inhibitory effect than the A8 peptide. In addition, a co-IP analysis was performed between rHA-mS100A8 (or rHis-mS100A9) and TLR4 or RAGE with increasing concentrations of S100A8 or A9 peptides. Consistent with the results of the fluorescence binding assay, when the amount of S100A8 or A9 peptide was increased, the rS100A8 or A9-TLR4 (or RAGE) interaction was inhibited (FIG. 2C). This demonstrated that the S100A8 or A9 peptide may compete with full-length S100A8 or A9 for binding to TLR4 or RAGE.

Next, it was evaluated whether A8 or A9 peptides may block the endogenous binding of S100A8 or A9 with TLR4 or RAGE in phagocytes. LPS treatment of immune cells increased the expression of PRRs such as TLR4 and RAGE and the secretion of S100 proteins. Therefore, an experiment was performed under LPS treatment conditions. Specifically, LPS-treated THP-1 or BMDMs were exposed to A8 or A9 peptides and then subjected to co-IP with TLR4 or RAGE (FIG. 2D). The results showed that S100A8 or A9-TLR4 (or RAGE) interaction was increased in the LPS-primed phagocytes (THP-1 and BMDM), while the intracellular expression levels remained unchanged. In particular, it was observed that the endogenous binding of S100 to the receptor was inhibited in both LPS-primed THP-1 and BMDMs upon treatment with the A8 or A9 peptide (FIG. 2D). Moreover, the S100-receptor interaction was specifically co-precipitated in human monocyte THP-1 and mouse BMDMs, indicating that the inhibitory effect of the peptide is preserved in both humans and mice.

In summary, the obtained results indicate that the A8 or A9 peptide including TLR4 and RAGE interaction motifs may successfully inhibit the binding of S100 to TLR4 or RAGE. These findings suggest that the peptides derived from S100 proteins have the potential to regulate inflammation.

### 2.3 S100A8/A9 Peptide Inhibits Activation of NLRP3 Inflammasome.

The NLRP3 inflammasome, a subset of cytoplasmic protein complexes, act importantly in sensing pathogens and initiating inflammatory responses under both physiological and pathological conditions. Unregulated activation of the NLRP3 inflammasome is related with IBD onset.

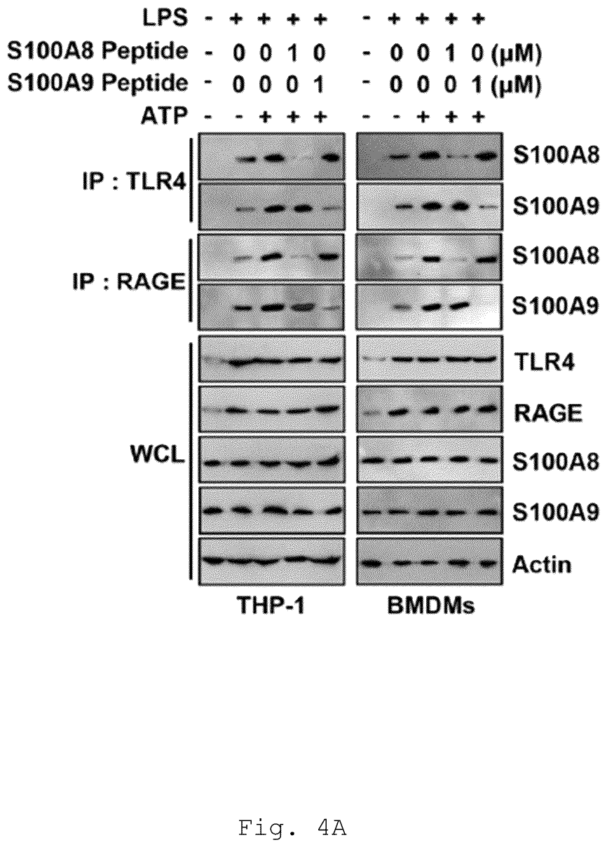

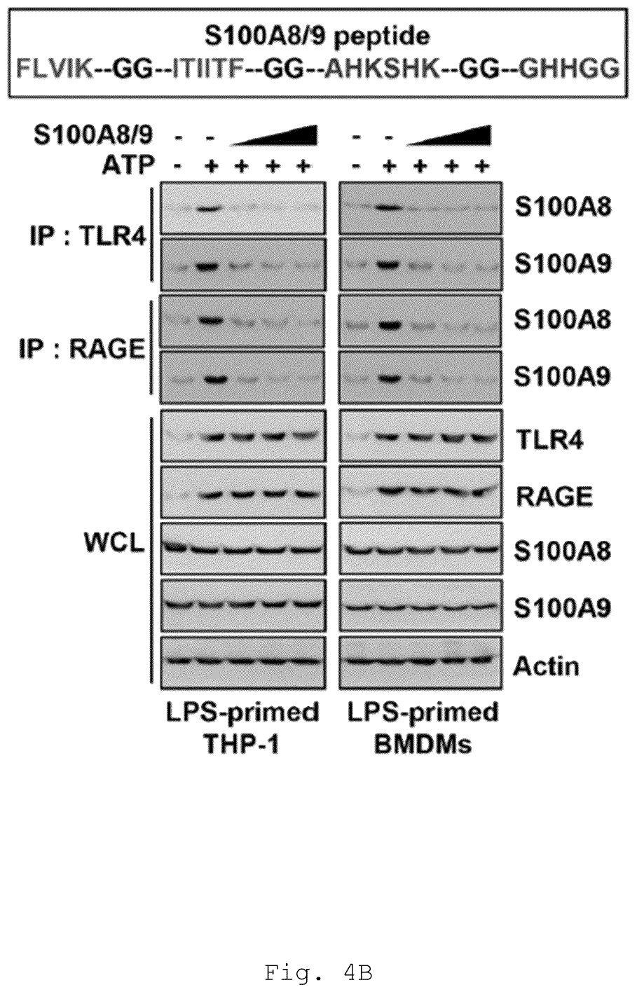

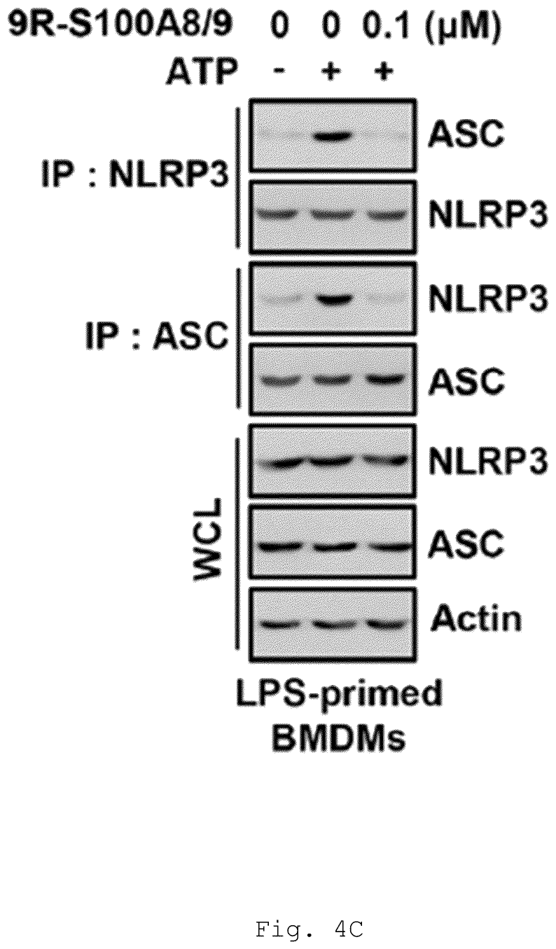

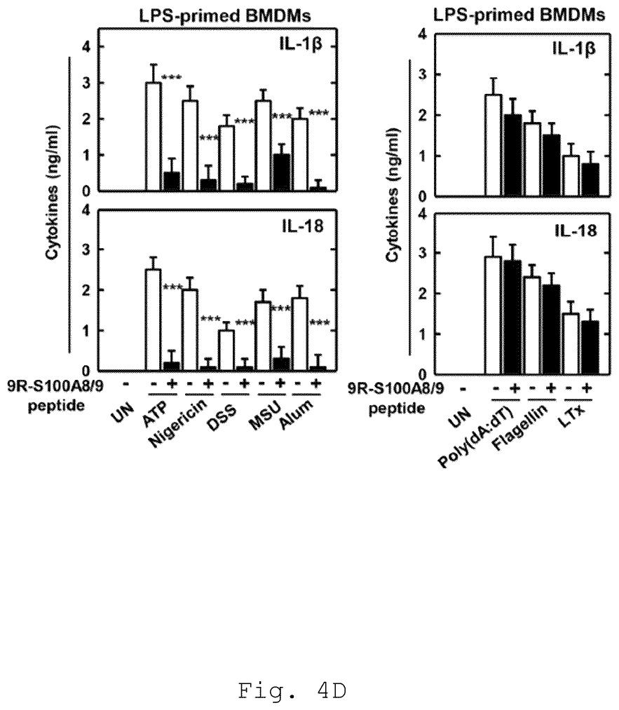

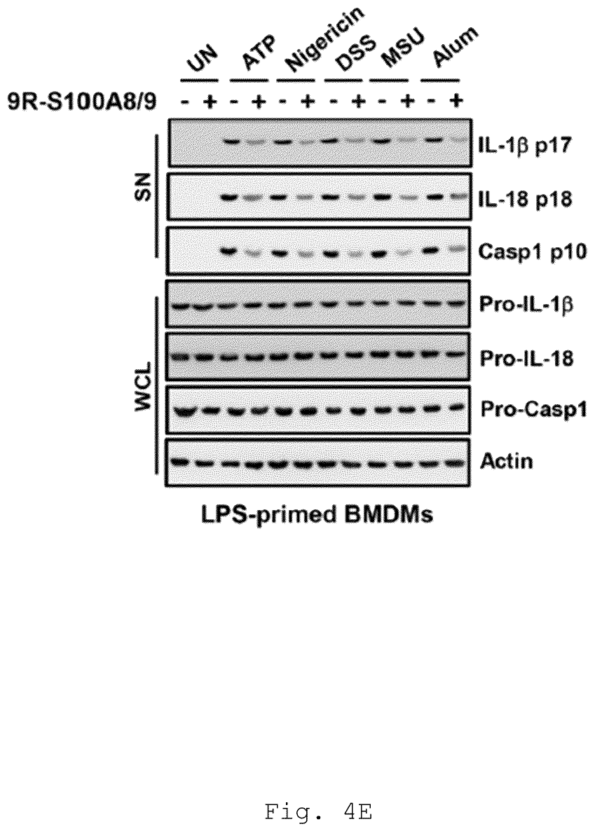

To investigate the role of S100A8 (or S100A9) in NLRP3 inflammasome activation, it was investigated whether the interaction of S100A8 (or S100A9) with TLR4 or RAGE is involved in NLRP3 inflammasome activation. It was found that S100A8 (or S100A9), expressed by an NLRP3 inflammasome inducer (LPS/ATP), increases extracellular S100 secretion and significantly increases the interaction of S100 with TLR4 and RAGE in both THP-1 and BMDMs. It was also found that this interaction is successfully inhibited by the A8 or A9 peptide (FIG. 4A).

Next, to improve the efficacy of the peptide, a multifaceted S100A8/A9 peptide including (bound to) polypeptides of an S100A8 or A9-derived TLR4 inhibitory motif (FLVIKG-GG-ITIITF) and an RAGE inhibitory motif (AHKSHK-GG-GHHGG) was designed (FIG. 4B, top). In LPS-primed phagocytes treated with ATP, the S100A8/A9 peptide showed higher inhibitory activity against extracellular binding of the S100-TLR4/RAGE interaction than the A8 or A9 peptide alone (FIG. 4B). In biological tests, the S100A8/A9 peptide, which is a dual TLR4/RAGE inhibitor, was tested in vitro at a concentration of 0.1 μM.

Next, to assess the effect of S100A8/A9 peptide on the activation of the NLRP3 inflammasome, NLPR3-ASC complex formation, caspase-1 activation, IL-1β and IL-18 production, and maturation and secretion of pro-caspase-1 were investigated. Pro-IL-1β/IL-18 plays an important role in inflammasome activation and IBD pathogenesis.

As can be seen from FIG. 4C, the co-IP analysis showed that 0.1 μM 9R-S100A8/A9 peptide successfully inhibited NLPR3-ASC interaction in ATP-stimulated LPS-primed BMDMs. To further investigate whether the S100A8/A9 peptide is associated with various NLRP3 inflammasome activators, including ATP, nigericin, DSS, MSU, aluminum (alum), and other inflammasomes, namely absent in melanoma 2 (AIM2) [activated by poly(dA:dT)], the present inventors treated LPS-primed BMDMs with the S100A8/A9 peptide and various inflammasome stimuli in NLR Family CARD Domain Containing 4 (NLRC4) (activated by Salmonella flagellin), and NLRP1 (activated by Bacillus anthracis toxin) inflammasomes. When treated with the S100A8/A9 peptide, the secretion levels of IL-1β and IL-18 were significantly reduced in response to NLRP3 activators, but the levels of AIM2, NLRC4, or NLRP1 activators were not affected in the LPS-primed BMDMs (FIG. 4D). This indicates that the 9R-S100A8/A9 peptide specifically regulates NLRP3 inflammasome activation. Furthermore, treatment with the 9R-S100A8/A9 peptide significantly reduced the production of caspase-1, IL-1β, and IL-18 in response to various NLRP3 inflammasome activators in the LPS-primed BMDMs. IL-1β, pro-IL-18, and pro-Caspase-1 derived from the priming step of NLRP3 inflammasome were not changed (FIG. 4E). This suggests that the S100A8/A9 peptide may be involved in the activation step of NLRP3 inflammasome activation rather than the priming step.

In conclusion, these results indicate that the S100A8/A9 peptide has the potential to regulate the NLRP3 inflammasome pathway by inhibiting the binding of S100A8 or S100A9 to TLR4 and RAGE. Consequently, these findings suggest that the S100A8/A9 peptide may have therapeutic potential for treating chronic inflammatory diseases, including IBD.

2.4. In Vitro Stability, PK, and Targeting Effect of rCT-S100A8/A9 on Inflamed Colon.

According to the results presented above, the A8/A9 peptide directly blocks the binding of S100A8 or A9 to extracellular TLR4 and RAGE, and at the same time, attenuates the activation of the NLRP3 inflammasome. This suggests the A8/A9 peptide has a potential role in regulating inflammation in colitis. Therefore, the present inventors further investigated whether this peptide has a protective effect in DSS-induced acute and chronic colitis models.

First, a multifaceted recombinant CT-S100A8/A9 protein including a combination of a colon-targeting peptide (TWYKIAFQRNRK (SEQ ID NO: 1), indicated as CT) and the S100A8 and A9 peptides was constructed (FIG. 5A). As confirmed in a previous study, the CT peptide binds to integrin α6β1, which is highly expressed in the colon, and is specifically delivered to the colon as a carrier for colonic delivery. The recombinant protein according to the present invention was found to have no significant effect on cell viability (FIG. 5A).

Second, the in vitro metabolic stability and PK parameters of rCT-S100A8/A9 were investigated, and the results are summarized in FIG. 5B. rCT-S100A8/A9 exhibited excellent plasma and microsomal stability, with 100% and 92% remaining 30 minutes after treatment with rCT-S100A8/A9, PBS, human plasma, and human liver microsomes, respectively. In addition, the inhibitory activity of rCT-S100A8/A9 against the five most abundant CYP isoenzymes was evaluated to investigate the toxicity of drug-drug interactions. The results showed that rCT-S100A8/A9 did not significantly reduce the activities of the tested CYP isoenzymes. In addition, the S100A8/A9 peptide exhibited similar effects.

Third, the PK parameters were observed when rCT-S100A8/A9 was administered intraperitoneally to female C57BL/6 mice. The AUC values and half-life indicated that rCT-S100A8/A9 was well maintained in the blood at effective drug concentrations, suggesting that rCT-S100A8/A9 is suitable for in vivo experiments compared to the S100A8/A9 peptide. Overall, the present inventors confirmed that rCT-S100A8/A9 has appropriate drug-like properties, including not only in vitro stability and PK characteristics but also pharmacological activity

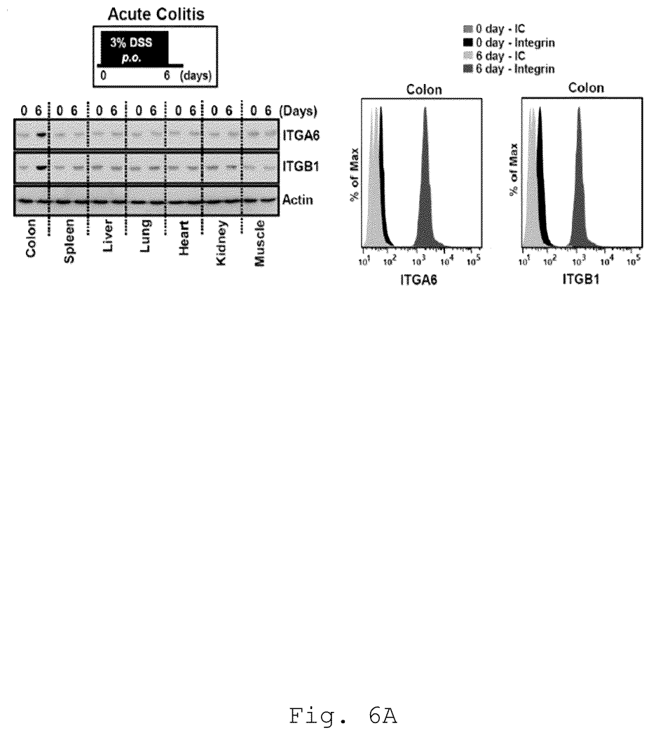

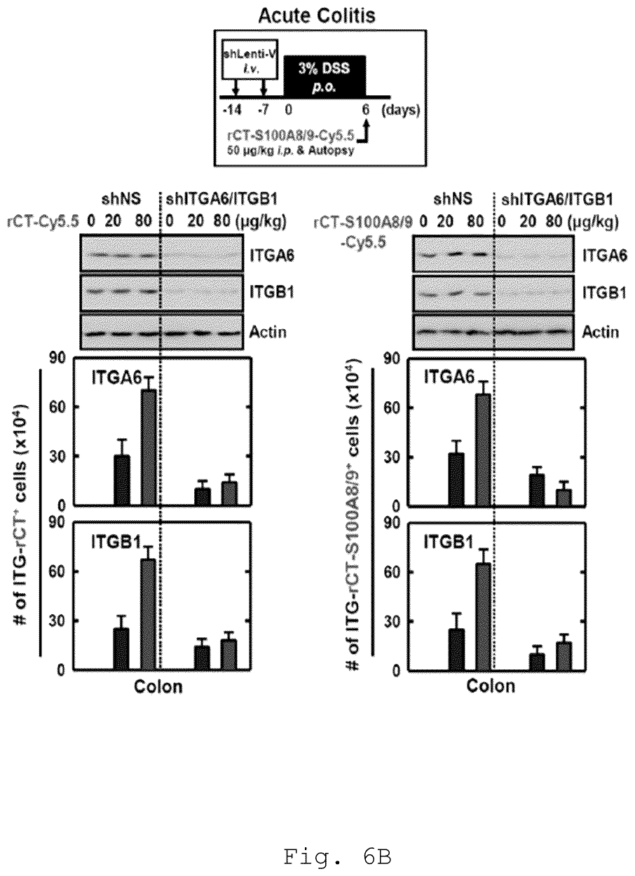

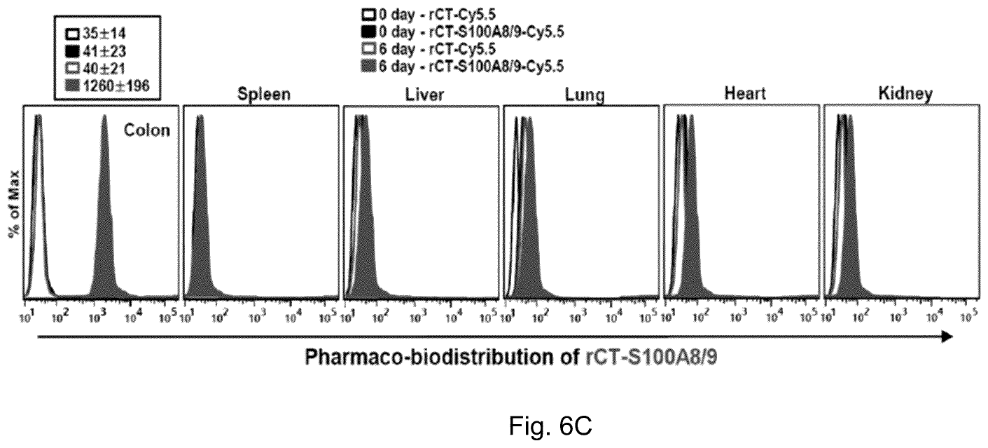

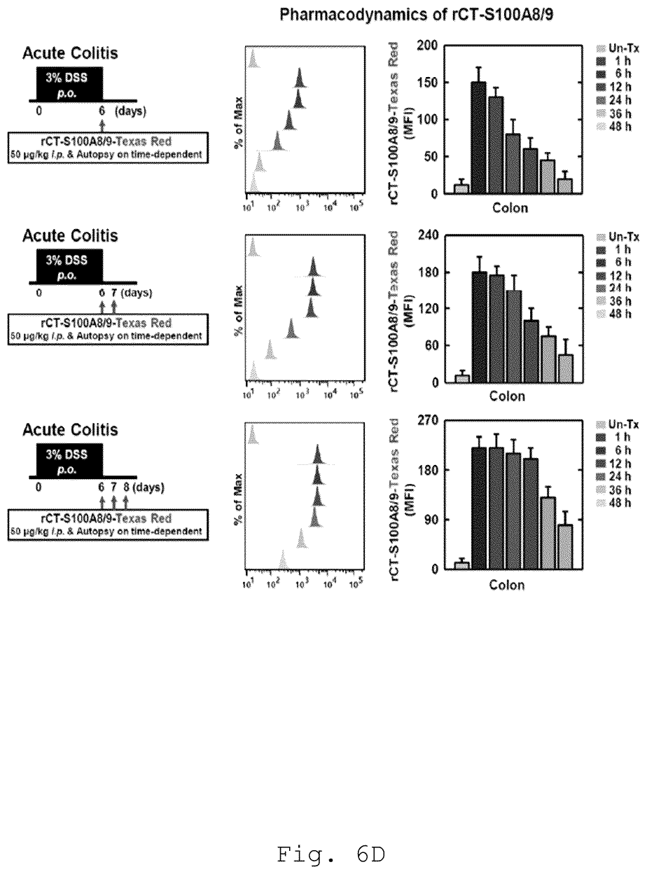

Fourth, to further investigate the physiological significance of rCT-S100A8/A9 in inflammatory colitis, a DSS-induced colitis mouse model was used (FIG. 6). It was found that the expression of ITGA6 (integrin a6) and ITGB1 (integrin 01), which bind to the CT peptide, was substantially increased in the colons of mice with acute colitis (FIG. 6A). Next, to evaluate the specificity of rCT-S100A8/A9, ITGA6- or ITGB1-knockdown mice were generated by sh-lentivirus transduction. The mice were treated with DSS, and rCT-S100A8/A9-Cy5.5 was administered intraperitoneally on day 6. The rCT or rCT-S100A8/A9 specifically targeted the colon tissues of the mice with acute colitis but not the other mice (FIGS. 6B and 6C). These results demonstrated that rCT binds to colonic tissues, suggesting the possibility of designing a colon-targeted drug delivery system for pharmaceutical applications to treat DSS-induced colitis. Finally, the pharmacodynamics of rCT-S100A8/A9 were evaluated, and according to the FACS analysis, 50% of the rCT-S100A8/A9 was retained in the colon when administered for three consecutive days to the mice with DSS-induced acute colitis (FIG. 6D).

2.5. Blockage of Exogenous S100A8/A9 with rCT-S100A8/A9 Ameliorates the Progression of Acute and Chronic Colitis.

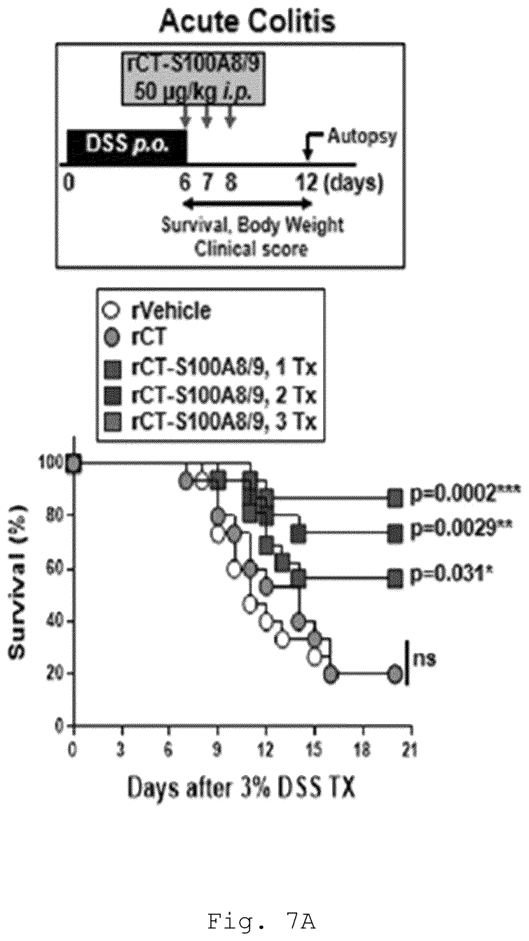

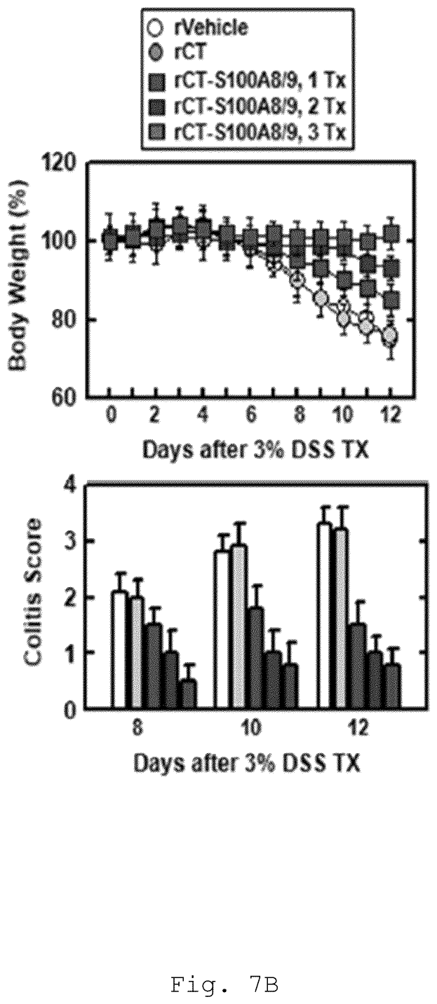

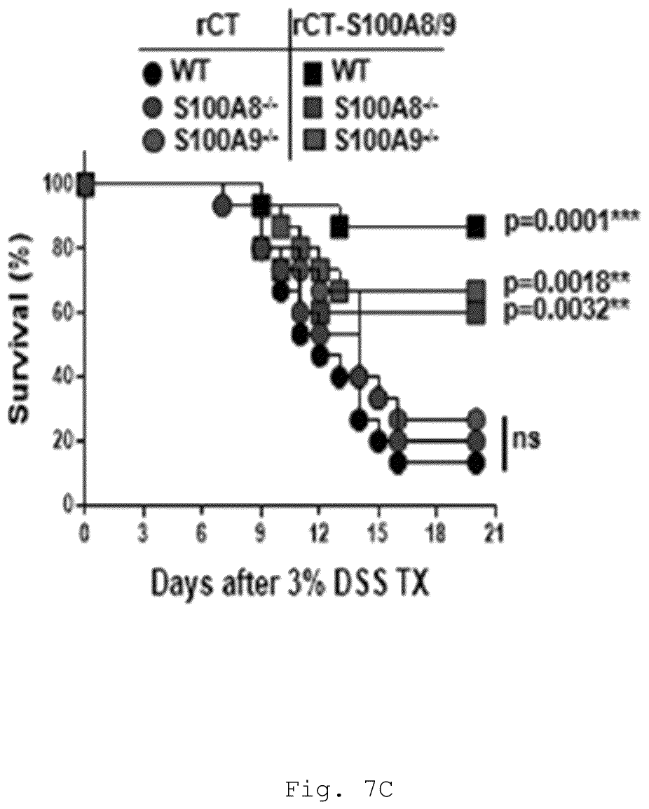

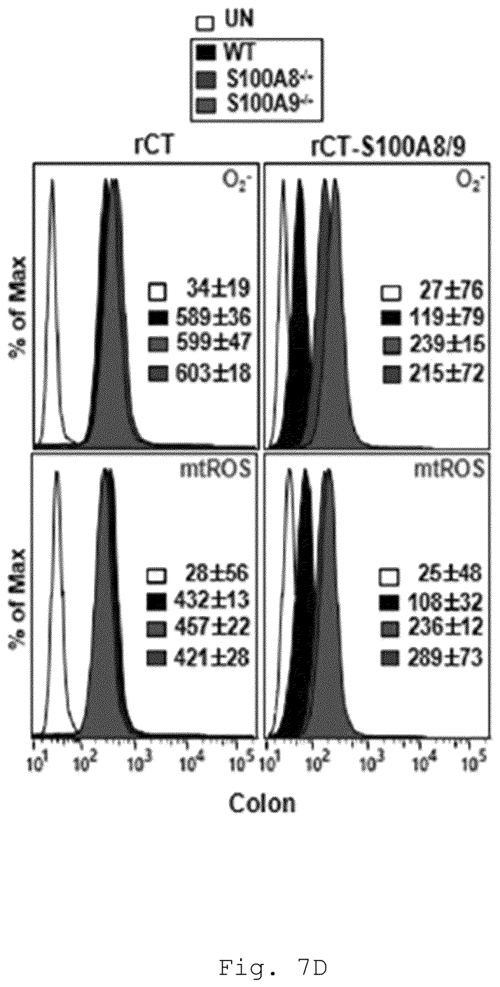

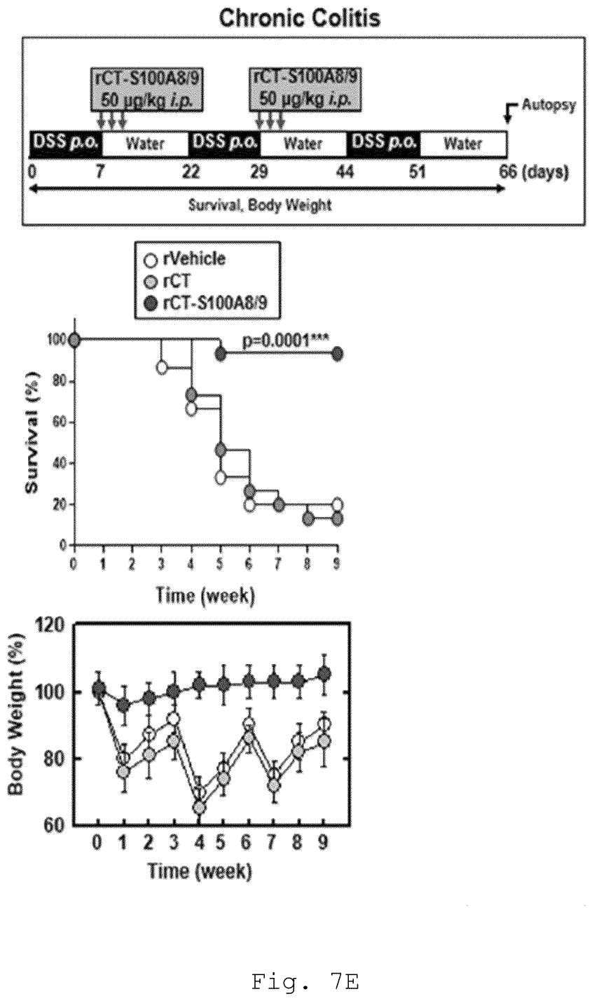

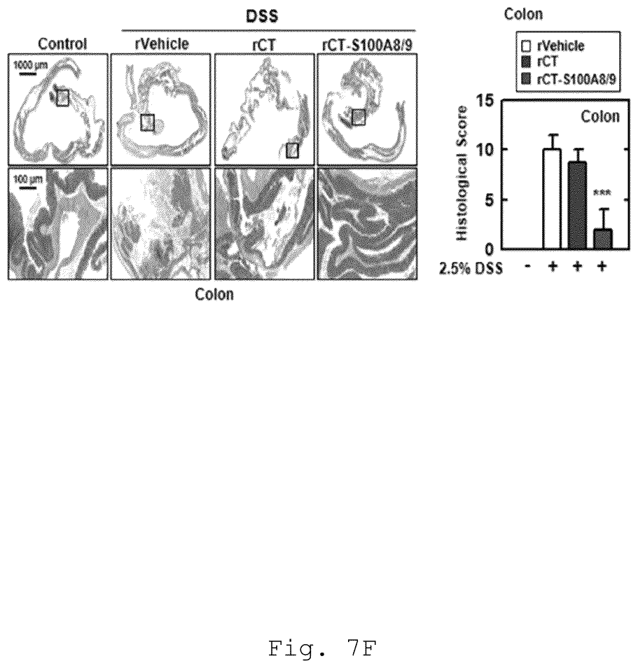

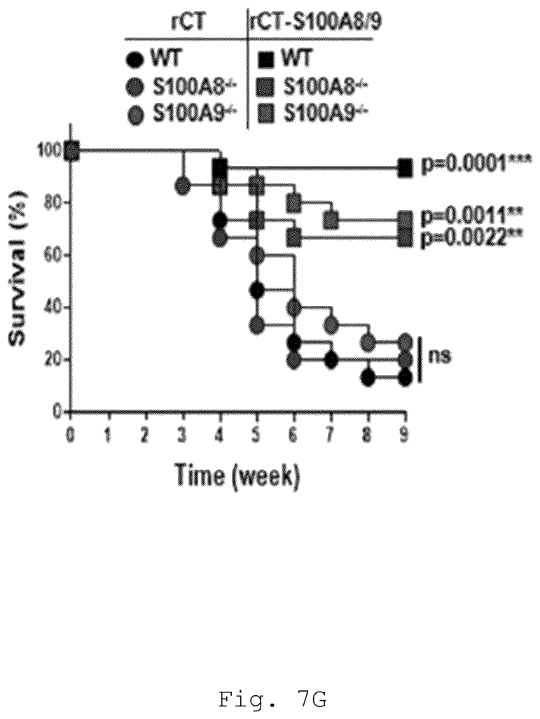

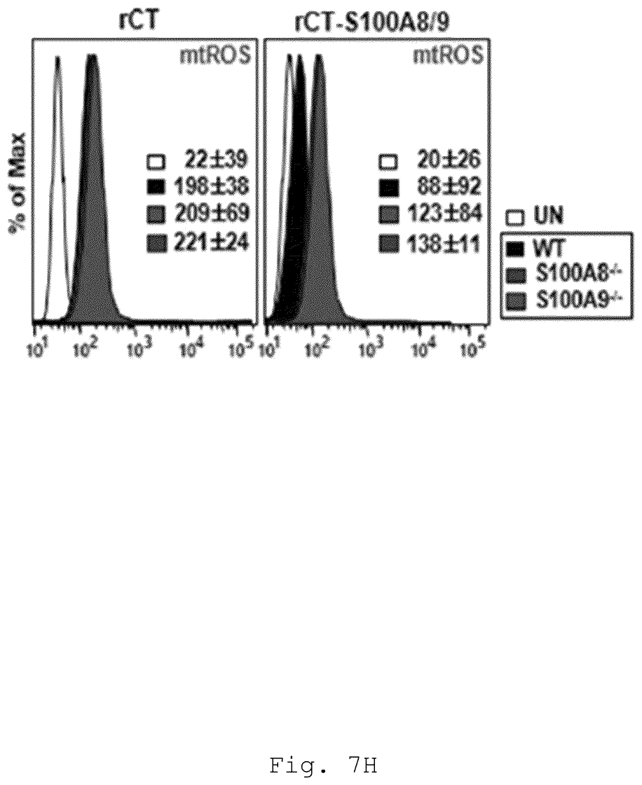

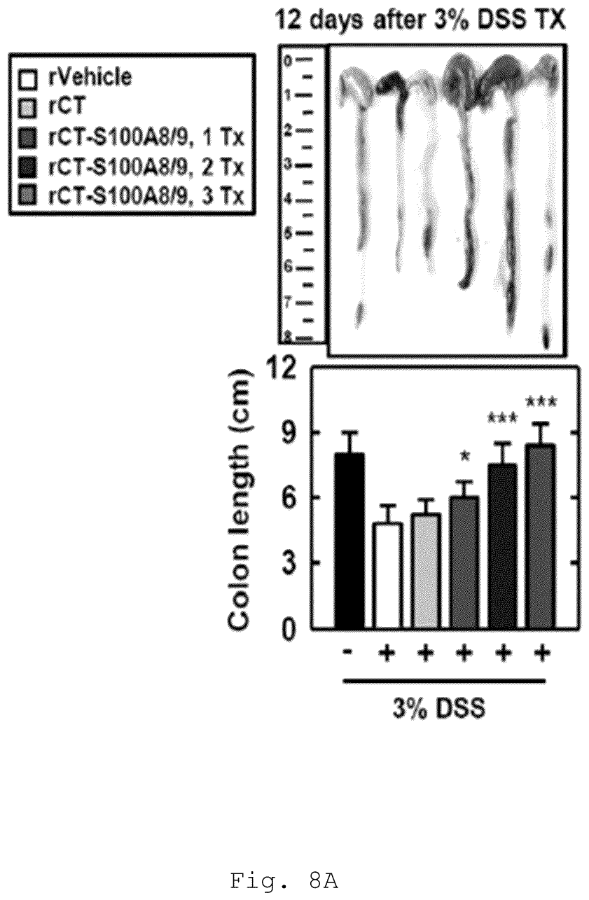

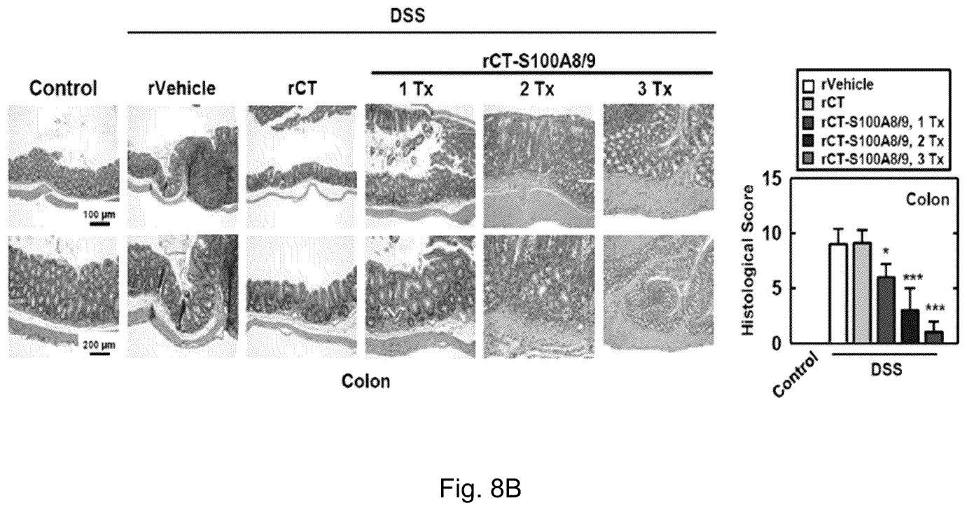

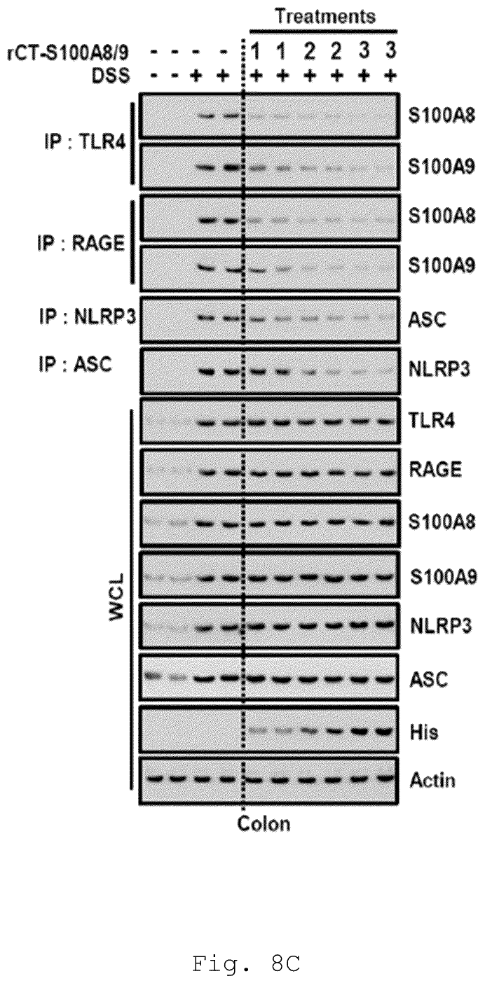

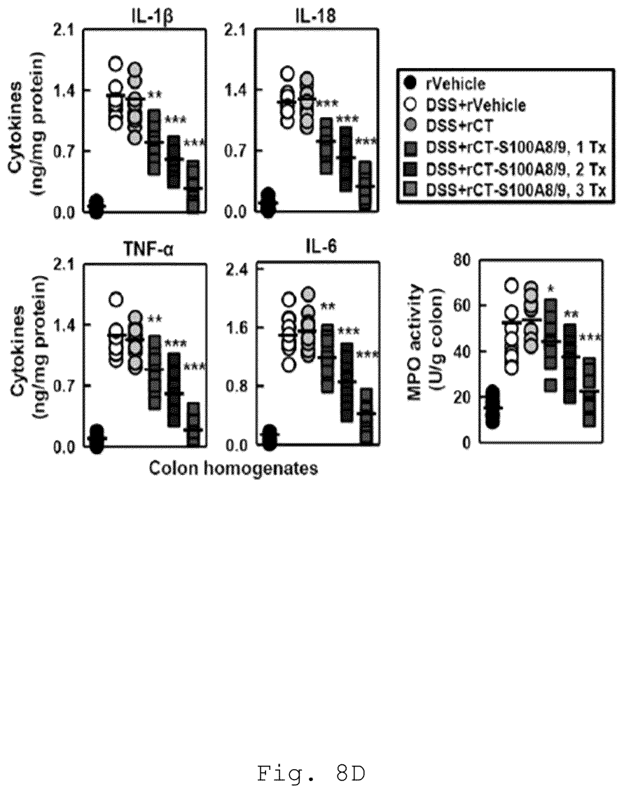

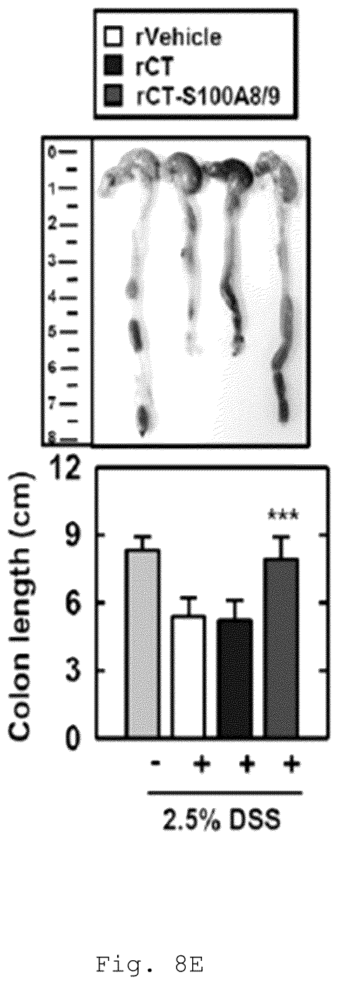

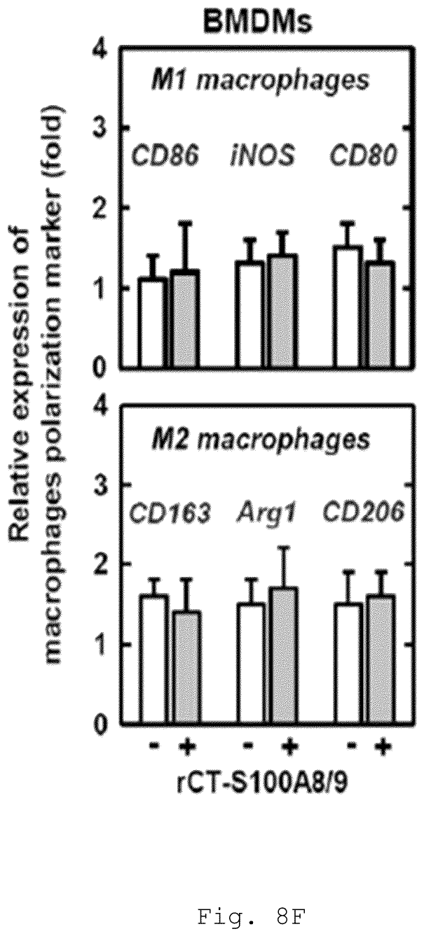

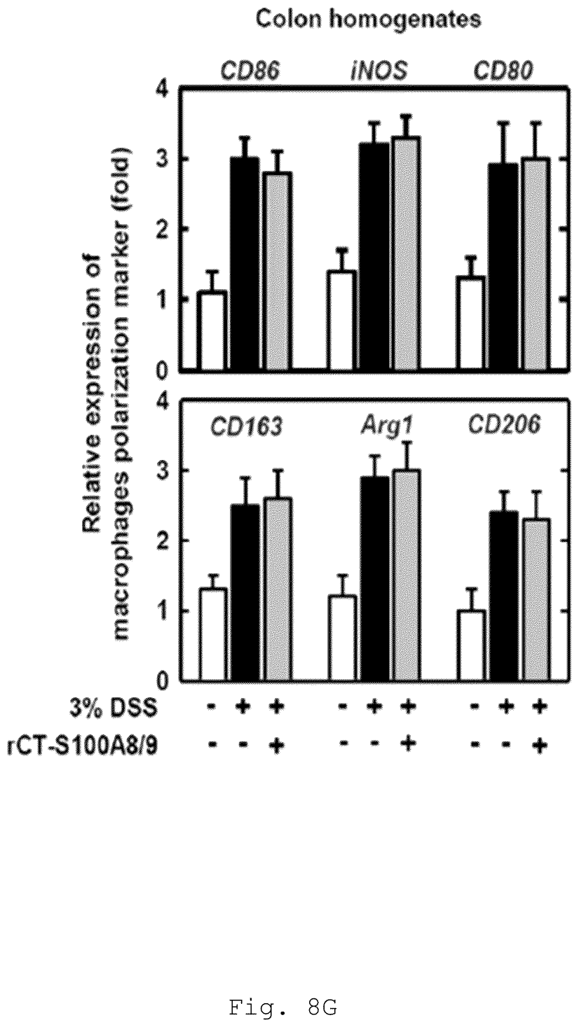

The therapeutic efficacy of rCT-S100A8/A9 on DSS-induced acute colitis in mice was evaluated. After colitis was induced by DSS, the mice were intraperitoneally injected with rCT-S100A8/A9 (50 μg/kg) once daily for 6, 7, or 8 days, and sacrificed on day 12 to evaluate the effect of treatment frequency (FIG. 7A, top). The results showed that rCT-S100A8/A9 significantly increased the survival rate of the mice with DSS-induced colitis in a treatment frequency-dependent manner (FIG. 7B, bottom). Consistent with the pharmacological study (FIG. 6D), the group that received treatment for three consecutive days showed the highest survival rate. In addition, the rCT-S100A8/A9-treated group showed less body weight loss and lower colitis scores than the control group (rVehicle or rCT-treated group) (FIG. 7B). Next, the length of the colon, which is an indicator of colitis, was evaluated. Colon length was recovered in proportion to the number of rCT-S100A8/A9 treatments (FIG. 8A). These results indicate that colon damage was reduced according to the frequency of rCT-S100A8/A9 treatment, as indicated by increased H&E staining (FIG. 8B).

To determine whether rCT-S100A8/A9 exerts pharmacological activity in vivo, the receptor-S100 interaction and NLRP3 inflammasome activation were analyzed in the colons of the treatment and control groups. The TLR4 (or RAGE)-S100A8 (or S100A9) and NLRP3-ASC interactions increased upon DSS treatment, but decreased after r-CT-S100A8/A9 treatment, showing a trend depending on the administration frequency (FIG. 8C).

In addition, the levels of pro-inflammatory cytokines (TNF-α, IL-1β, IL-6, and IL-18) and the activity of myeloperoxidase (MPO) involved in cytokine production in colonic homogenates were investigated (FIG. 8D). The results showed that the levels of pro-inflammatory cytokines and MPO activity in colonic homogenates of mice were significantly reduced upon rCT-S100A8/A9 treatment. In particular, it was found through S100A8 KO and S100A9 KO experiments that rCT-S100A8/A9 specifically regulated acute colitis survival (FIG. 7C) and ROS (cellular ROS and mtROS) (FIG. 7D) in an S100A8- and S100A9-dependent manner.

Next, the therapeutic effect of rCT-S100A8/A9 on chronic colitis was confirmed. Chronic DSS colitis was induced by three cycles of DSS+water combination, and intraperitoneal rCT-S100A8/A9 injection was performed once a day for three consecutive days of DSS administration in each cycle. Similar to the acute colitis model, the rCT-S100A8/A9 group showed a pattern of significantly reduced body weight loss (FIG. 7E). Then, the length of the colon, which is an indicator of colitis, was measured, and the colon length was recovered in the rCT-S100A8/A9 treatment group (FIG. 8E). These changes showed that colon damage was decreased, as indicated by increased H&E staining (FIG. 5F). Consistent with acute colitis, it was found that rCT-S100A8/A specifically regulated chronic colitis survival (FIG. 7G) and ROS (FIG. 7H) in an S100A8- and S100A9-dependent manner. However, macrophage polarization by rCT-S100A8/A9 was not affected in macrophages (FIG. 8F) and colonic lysates (FIG. 8G).

The experimental results showed that treatment with rCT-S100A8/A9 reduced acute and chronic inflammation in the colon by blocking TLR4 and RAGE and regulating the NLRP3 inflammasome. These findings highlight the need for further investigation of rCT-S100A8/A9 as a candidate therapeutic for IBD.

2.6. rCT-S100A8/A9 Attenuated Tumor Formation in the CAC Model.

There are increasing reports that chronic inflammation increases the risk of tumorigenesis. Therefore, the therapeutic efficacy of rCT-S100A8/A9 was evaluated in a CAC mouse model to determine whether the progression of the “inflammation-cancer link” is inhibited by inhibiting S100-PRRs interactions.

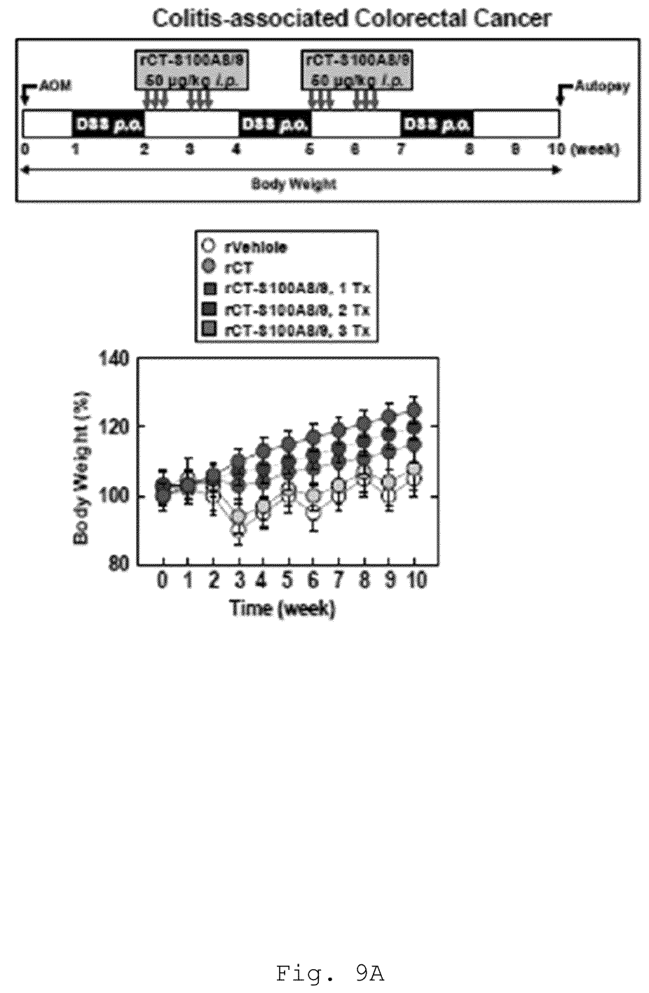

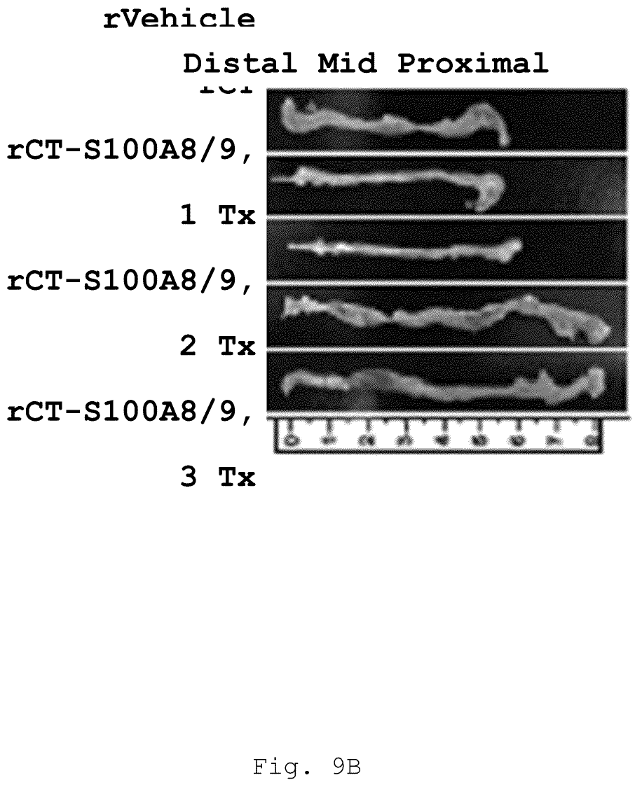

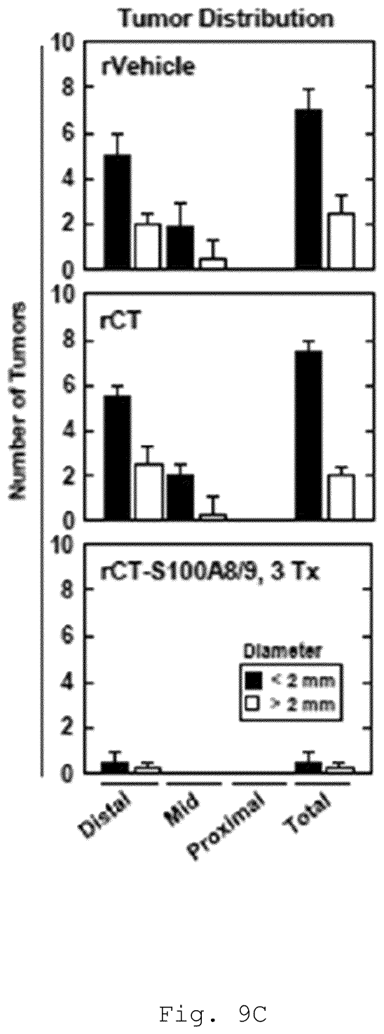

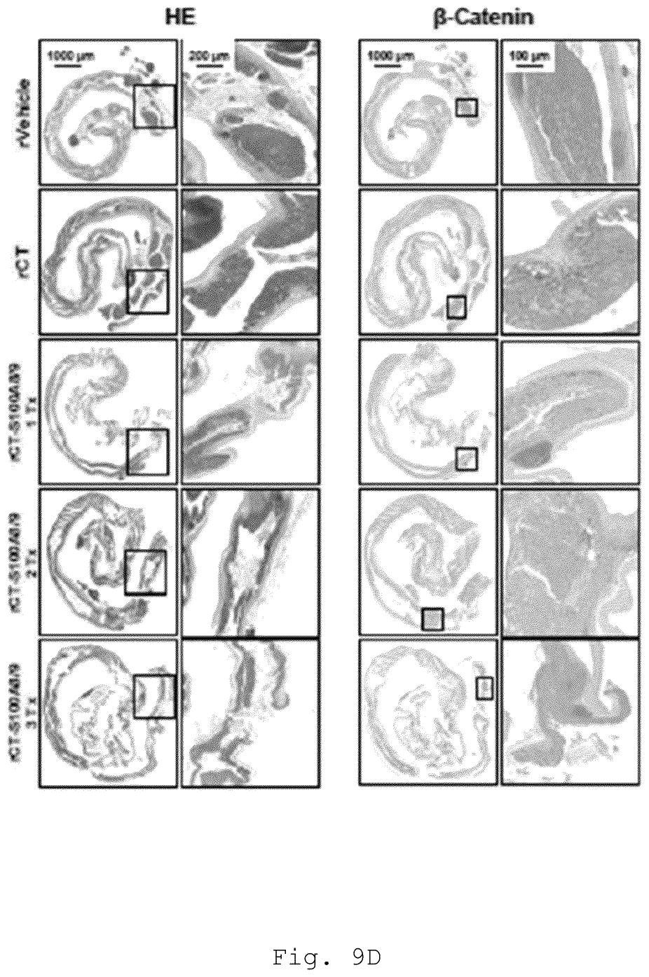

AOM/DSS-treated mice were administered rCT-S100A8/A9 four times according to the administration frequency during three DSS cycles until week 10 (FIG. 9A, top). The body weights of CRC mice treated with rVehicle or rCT showed a low variability in weight gain, whereas the mice treated with rCT-S100A8/A9 showed a higher weight gain rate according to the treatment frequency, which was more prominent with the weight gain (FIG. 9A). Tumor induction by AOM/DSS treatment was mainly observed in the distal colon and produced colonic shortening in the CAC mouse model. As shown in FIG. 9B, the control groups (rVehicle and rCT) showed high tumor burden in the distal colon along with short colonic lengths. However, in the rCT-S100A8/A9-treated group, the colonic length was restored, and the number of tumors less than 2 mm was significantly reduced in the group that received rCT-S100A8/A9 treatment for three consecutive days.

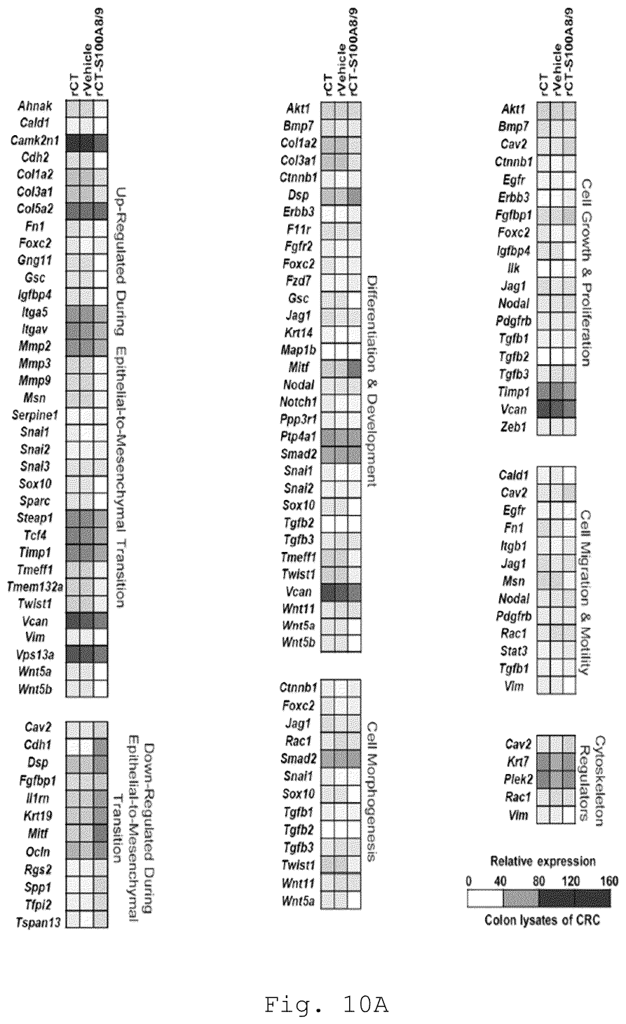

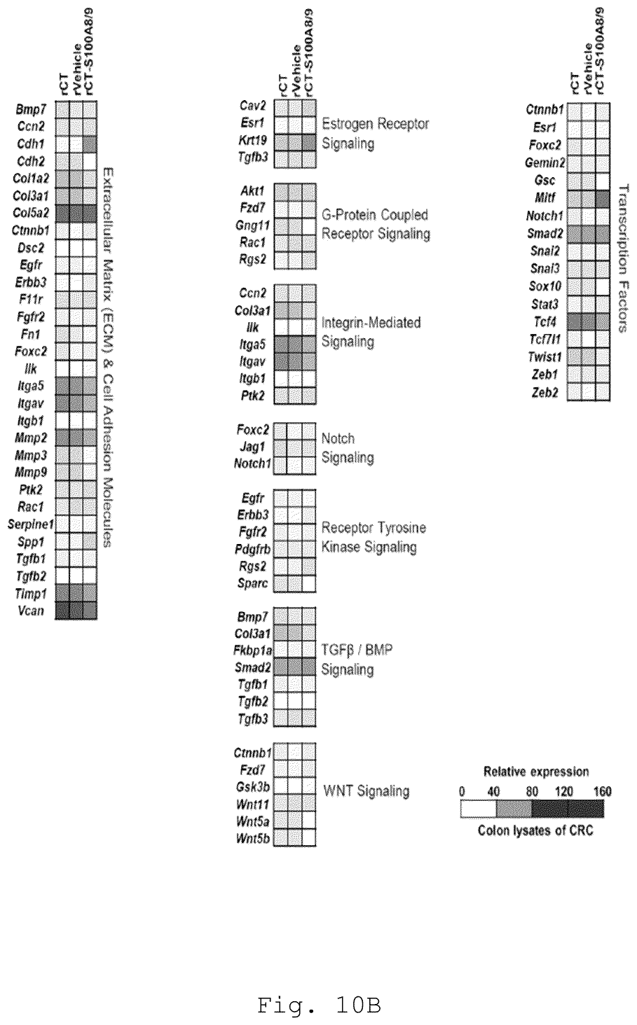

To further evaluate the colitis-associated tumors, H&E staining was performed. A histological analysis of colon tissues obtained from AOM/DSS-induced CRC mice showed the presence of severe inflammation, characterized by mucosal damage, surface ulceration, and destruction of the foveolar epithelium. However, administration of rCT-S100A8/A9 demonstrated a significant alleviation of histological colonic damage, especially in terms of inflammatory infiltration and epithelial damage, as indicated by the preferential effect of histological scores (FIG. 9D, left). Immunohistochemical analysis showed that β-catenin was significantly upregulated in colonic adenocarcinoma cells of CAC mice compared with normal tissues. As expected (data not shown), when treated with rCT-S100A8/A9, the protein levels of these markers were effectively suppressed (FIG. 9D, right). In addition, a profiler PCR array was performed on CRC colon tissues to confirm the signaling mechanisms and molecular targets regulated by rCT-S100A8/A9, and it was confirmed that epithelial-to-mesenchymal transition was regulated (FIG. 10A-10B). In summary, these observations indicate that rCT-S100A8/A9 treatment is effective in alleviating the progression of CAC.

2.7. rCT-S100A8/A9 Inhibited Oxaliplatin-Resistant HCT116 Xenograft Tumor Growth.

Oxaliplatin is an effective chemotherapeutic commonly used to treat CRC, but long-term use thereof often results in drug resistance. Based on the evidence that rCT-S100A8/A9 alleviates colitis and CRC in mouse models, experiments were conducted to investigate its efficacy in overcoming oxaliplatin resistance.

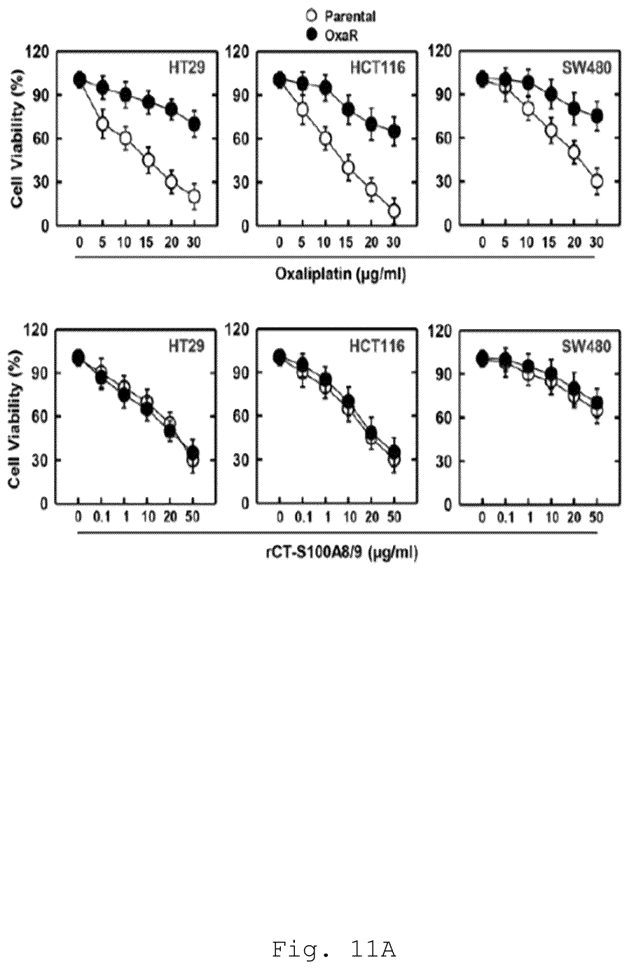

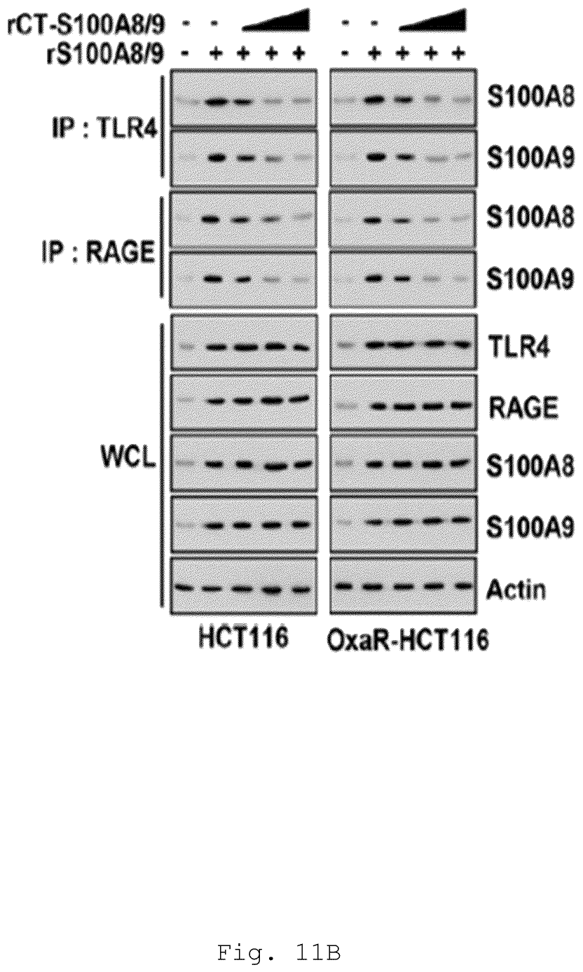

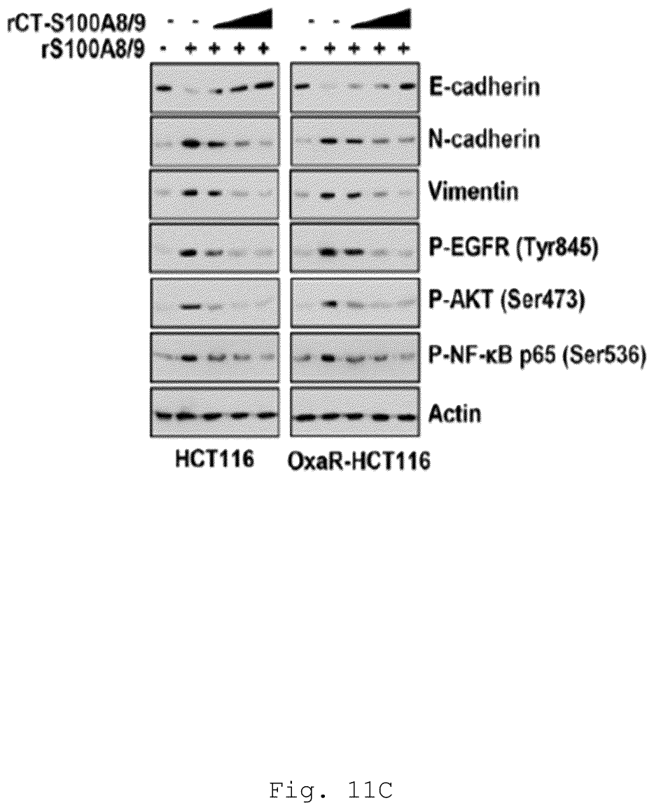

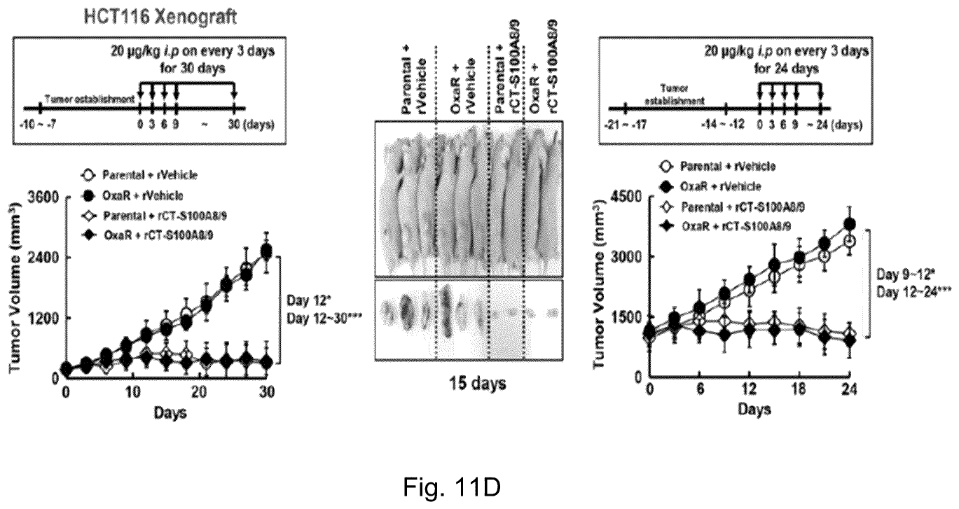

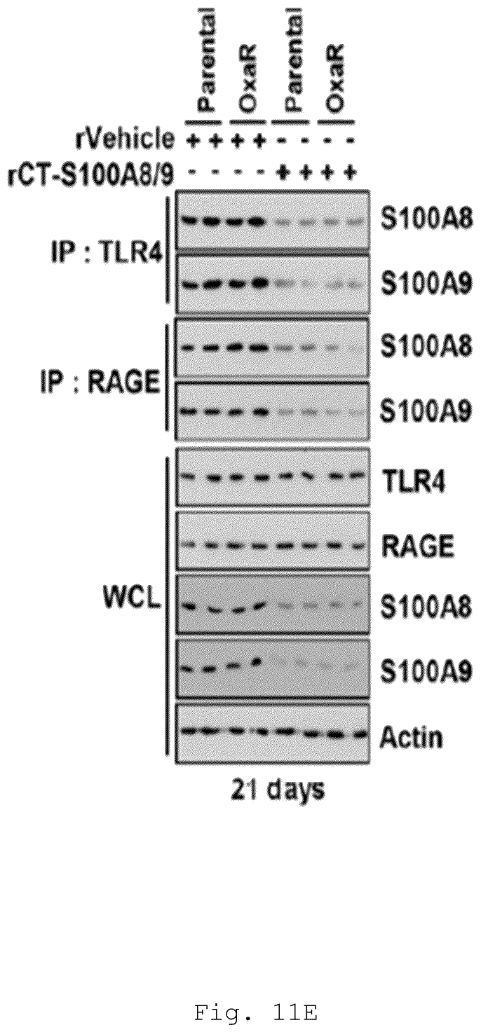

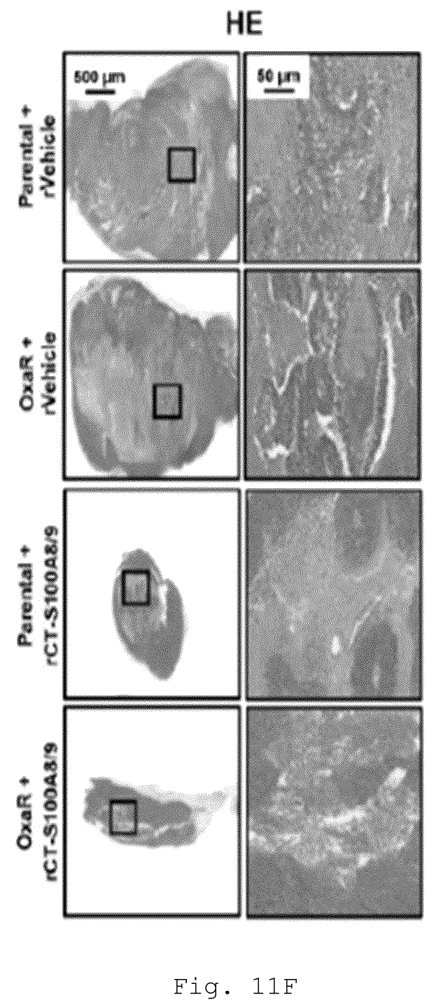

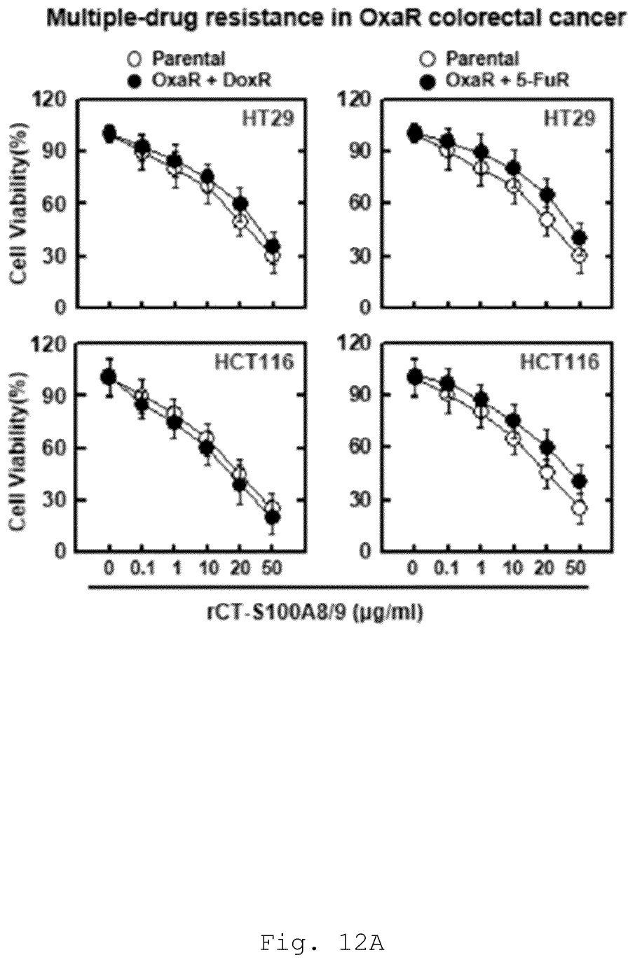

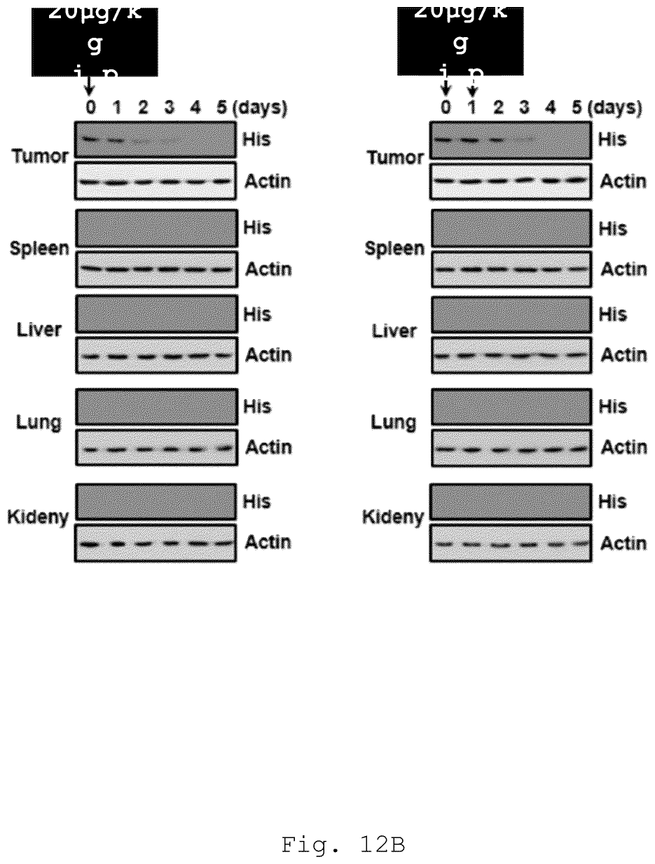

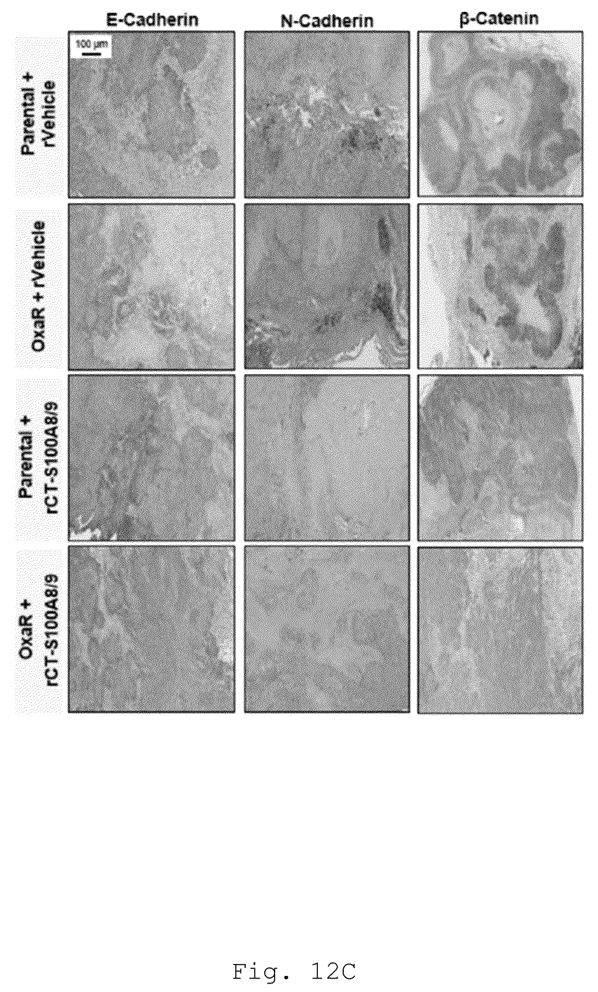

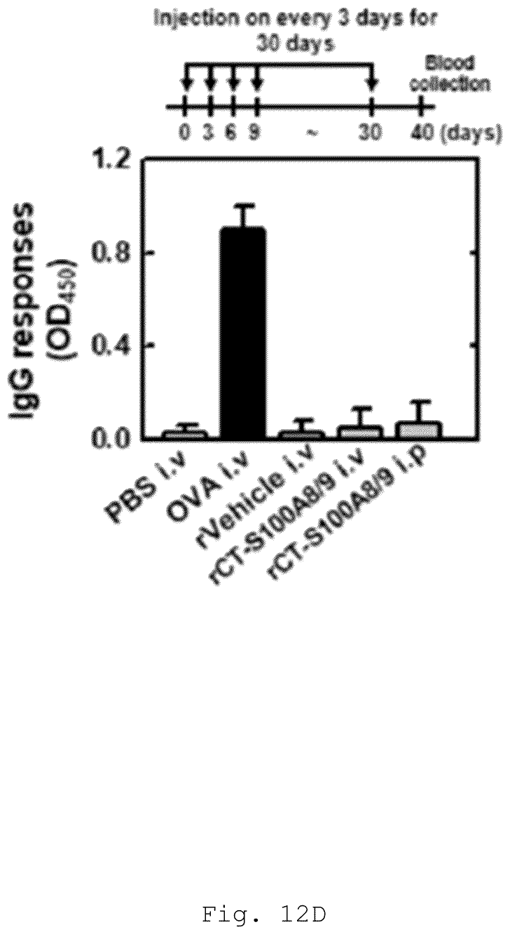

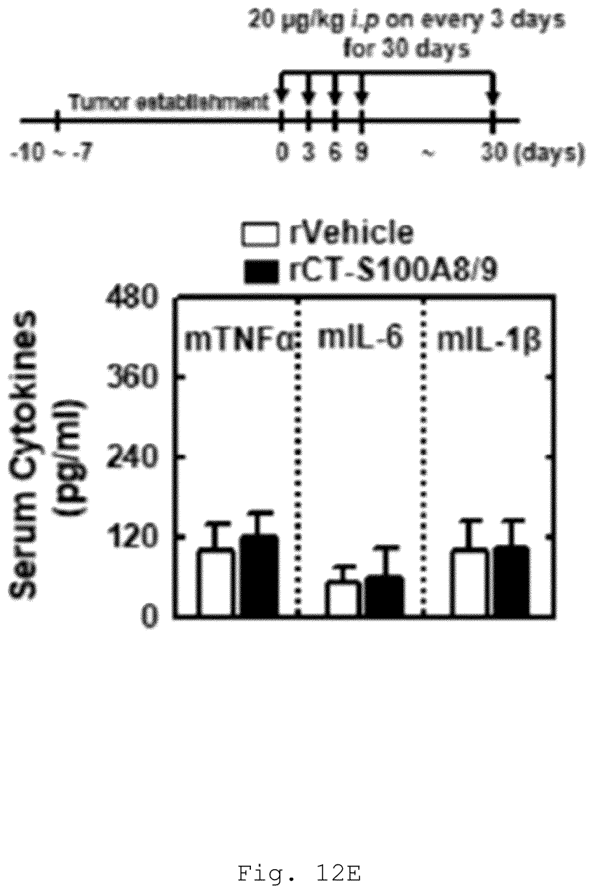

To confirm the inhibitory effect of rCT-S100A8/A9 on oxaliplatin resistance, the present inventors performed a water-soluble tetrazolium-8 (WST-8)-based cell viability assay using parental cells of three CRC cell lines, HT29, HCT-116 and SW480, and their corresponding oxaliplatin-resistant (OxaR) cell lines. OxaR CRC cells (HT29, HCT116 and SW480) were established by long-term exposure of CRC cells to oxaliplatin as described in Example 1. As shown in FIG. 11A, OxaR CRC cells exhibited significant resistance to oxaliplatin treatment. rCT-S100A8/A9 treatment not only reduced the viability of OxaR CRC cells, but also exhibited efficacy against multidrug resistance, including doxorubicin-resistant (DoxR) and 5-fluorouracil-resistant (5-FuR) CRC cells (FIG. 12A).

It was found that when the expression of TLR4 and RAGE was increased in colon cancer cells after treatment with 5-FU and oxaliplatin, cell viability was promoted and epithelial-to-mesenchymal transition (EMT) was induced through the activation of NF-κB signaling. Moreover, the extracellular binding of S100A8/A9 to these PRRs was found to induce the activation of NF-κB, thereby promoting tumor growth and chemoresistance. Accordingly, the present inventors inferred that the inhibitory effect of rCT-S100A8/A9 on OxaR CRC may result from the blockage of S100-PRR interactions. In OxaR-HCT116, the present inventors observed that recombinant S100A8/A9 increased not only the expression of TLR4/RAGE but also S100-PRR interactions, which were inhibited by increasing the concentration of rCT-S100A8/A9 (FIG. 11B). To confirm whether the blockage of S100-PRR interactions is correlated with the inhibitory effect on OxaR CRC, cells treated with rCT-S100A8/A9 were lysed, and a Western blot analysis was performed to investigate the expression of EMT markers. As can be seen in FIG. 11C, a significant decrease in the expression of mesenchymal markers (N-cadherin and vimentin) and an increase in the expression of an epithelial marker (E-cadherin) were observed in both parental cells and OxaR-HCT116 cells. In addition, the phosphorylation of epidermal growth factor receptor (EGFR), AKT, and NF-κB, which are involved in chemotherapy resistance pathways, was also decreased in both rCT-S100A8/A9-treated parental cells and OxaR CRC cells. These results suggest that targeting S100-PRR interactions by rCT-S100A8/A9 may be involved in the restoration of chemoresistance and exert cytotoxic effects on OxaR CRC.

To assess the antitumor efficacy of rCT-S100A8/A9, xenograft mouse models were established using parental cells or OxaR HCT116 cells. When rCT-S100A8/A9 was intraperitoneally injected at a dose of 20 μg/kg into the parental cells or OxaR HCT116-bearing mice, the presence of rCT-S100A8/A9 in tumor cells was detected by Western blot analysis. This localization of rCT-S100A8/A9 in tumor cells was observed to persist for up to two days after injection and then decreased on day 3 (FIG. 12B).

Next, tumor growth inhibition assays were performed under various tumor growth conditions, and rCT-S100A8/A9 treatment was started according to the size of the established tumors. For initial tumor growth, rCT-S100A8/A9 treatment was started 10 days after tumor establishment, and for actively growing tumors, rCT-S100A8/A9 treatment was started 21 days after tumor establishment. As expected, rCT-S100A8/A9 treatment significantly inhibited tumor growth in mice with both initially growing and actively growing tumors (FIG. 11D). The Western blot analysis results showed that rCT-S100A8/A9 treatment significantly reduced the interaction of S100 and PRRs in xenograft tumors (FIG. 11E). Consistent with these findings, immunostaining of tumor sections using EMT markers showed that the expression of E-cadherin was increased and the expression of N-cadherin and β-catenin was decreased in the rCT-S100A8/A9-treated xenografts compared to control group levels (FIGS. 11F and 12C).

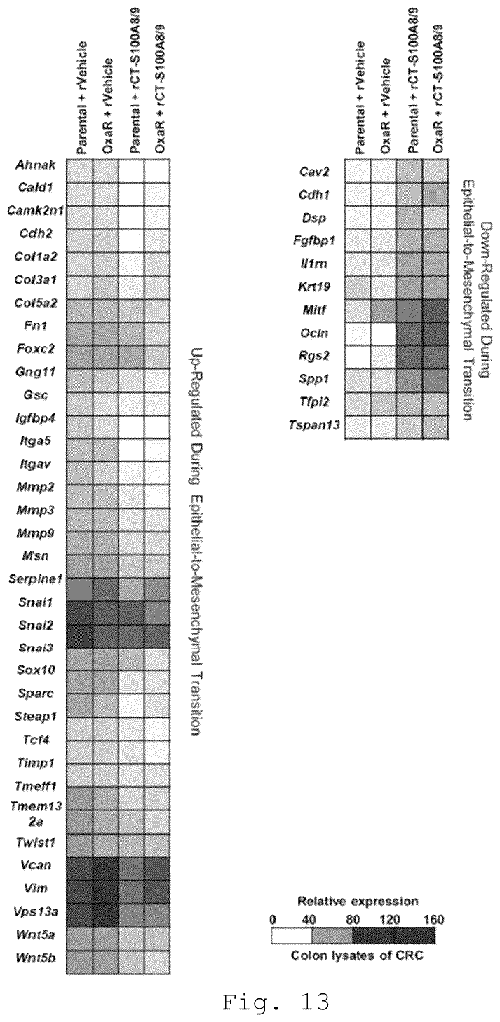

A major concern with protein therapeutics derived from endogenous human proteins is that they may induce strong anti-therapeutic antibody responses and stimulate undesirable immune responses. These may include immune-mediated side effects and potential loss of efficacy. Therefore, the immunogenicity of rCT-S100A8/A9 was assessed by measuring the production of anti-rCT-S100A8/A9 antibodies in mice immunized with the peptide according to the present invention. The production of specific anti-rCT-S100A8/A9 antibodies was negligible compared to that induced by ovalbumin (FIG. 12D). In addition, the production of pro-inflammatory cytokines induced by rCT-S100A8/A9 was negligible in tumor-bearing mice. There was no difference in the serum levels of TNF-α, IL-6, and IL-1β between the negative control and rCT-S100A8/A9-administered mice (FIG. 12E), indicating that rCT-S100A8/A9 induced insignificant immunogenicity and immune responses in BALB/c mice. In addition, it was confirmed by performing a profiler PCR array that, similar to CRC, rCT-S100A8/A9 regulates the involvement of epithelial-to-mesenchymal transition in tumors of HCT116 xenografts (FIG. 13).

### Advantageous Effects

The polypeptide according to the present invention has the ability to treat colitis or colorectal cancer.

For example, for purposes of claim construction, the claims set forth below should not be construed narrowly in any way compared to the literal language thereof, and thus exemplary embodiments from the specification should not be read as claims. Therefore, it should be understood that the present invention has been described by way of example and not as a limitation on the scope of the claims. Therefore, the present invention is limited only by the claims below. All publications, issued patents, patent applications, books, and journal articles cited in the present application are hereby respectively incorporated by reference in their entirety.

What is claimed is:

1. A fusion protein in which four peptides consisting of amino acid sequences represented by SEQ ID NO: 2 to SEQ ID NO: 5 are linked.

2. The fusion protein of claim 1, including an amino acid sequence represented by (SEQ ID NO: 2)-(linker)-(SEQ ID NO: 3)-(linker)-(SEQ ID NO: 4)-(linker)-(SEQ ID NO: 5).

3. The fusion protein of claim 2, wherein the linker is (G)ₙ, (A)ₙ, or (G₄S)ₘ, where m is an integer from 1 to 3, and n is an integer from 2 to 8.

4. The fusion protein of claim 1, wherein SEQ ID NO: 1 is further linked to an N-terminus or C-terminus.

5. The fusion protein of claim 1, including an amino acid sequence represented by (SEQ ID NO: 1)ₐ-(linker)-{(SEQ ID NO: 2)-(linker)-(SEQ ID NO: 3)-(linker)-(SEQ ID NO: 4)-(linker)-(SEQ ID NO: 5)}_{b}, wherein a is an integer from 1 to 7 and b is an integer from 1 to 5.

6. A fusion protein comprising SEQ ID NO: 6.

7. The fusion protein of claim 6, wherein SEQ ID NO: 1 is further linked to an N-terminus or C-terminus.

8. The fusion protein of claim 6, consisting of an amino acid sequence represented by SEQ ID NO: 7.

9. A method of treating colitis or colorectal cancer, comprising administering a fusion protein in which four peptides consisting of amino acid sequences represented by SEQ ID NO: 2 to SEQ ID NO: 5 are linked or a fusion protein including SEQ ID NO: 6 in a pharmaceutically effective amount to a patient in need of treatment.

10. The method of claim 9, wherein the fusion protein includes an amino acid sequence represented by (SEQ ID NO: 2)-(linker)-(SEQ ID NO: 3)-(linker)-(SEQ ID NO: 4)-(linker)-(SEQ ID NO: 5).

11. The method of claim 10, wherein the linker is (G)ₙ, (A)ₙ, or (G₄S)ₘ, where m is an integer from 1 to 3, and n is an integer from 2 to 8.

12. The method of claim 9, wherein SEQ ID NO: 1 is further linked to an N-terminus or C-terminus.

13. The method of claim 9, wherein the fusion protein includes an amino acid sequence represented by (SEQ ID NO: 1)ₐ-(linker)-{(SEQ ID NO: 2)-(linker)-(SEQ ID NO: 3)-(linker)-(SEQ ID NO: 4)-(linker)-(SEQ ID NO: 5)}_{b}, wherein a is an integer from 1 to 7 and b is an integer from 1 to 5.


SEQUENCE LISTING (14 sequences)

SEQ ID NO: 1 - protein, 12 aa, synthetic construct
TWYKIAFQRNRK

SEQ ID NO: 2 - protein, 5 aa, synthetic construct
FLVIK

SEQ ID NO: 3 - protein, 6 aa, synthetic construct
AHKSHK

SEQ ID NO: 4 - protein, 6 aa, synthetic construct
ITIITF

SEQ ID NO: 5 - protein, 5 aa, synthetic construct
GHHGG

SEQ ID NO: 6 - protein, 28 aa, synthetic construct
FLVIKGGAHKSHKGGITIITFGGGHHGG

SEQ ID NO: 7 - protein, 146 aa, synthetic construct
TWYKIAFQRNRKTWYKIAFQRNRKTWYKIAFQRNRKTWYKIAFQRNRKTWYKIAFQRNRKGGFLVIKGGAHKSHKGGITIITFGGGHHGGFLVIKGGAHKSHKGGITIITFGGGHHGGFLVIKGGAHKSHKGGITIITFGGGHHGG

SEQ ID NO: 8 - protein, 89 aa, Mus musculus
MPSELEKALSNLIDVYHNYSNIQGNHHALYKNDFKKMVTTECPQFVQNINIENLFRELDINSDNAINFEEFLAMVIKVGVASHKDSHKE

SEQ ID NO: 9 - protein, 113 aa, Mus musculus
MANKAPSQMERSITTIIDTFHQYSRKEGHPDTLSKKEFRQMVEAQLATFMKKEKRNEALINDIMEDLDTNQDNQLSFEECMMLMAKLIFACHEKLHENNPRGHGHSHGKGCGK

SEQ ID NO: 10 - protein, 5 aa, synthetic construct
GSGSG

SEQ ID NO: 11 - protein, 6 aa, synthetic construct
GSGSGG

SEQ ID NO: 12 - protein, 13 aa, synthetic construct
GSGGGGSGGGGSG

SEQ ID NO: 13 - protein, 5 aa, synthetic construct
GGGGS

SEQ ID NO: 14 - protein, 15 aa, synthetic construct
GGGGSGGGGSGGGGS